(19) United States
(12) Patent Application Publication
(10) Pub. No.: US 2011/0265226 A1
(43) Pub. Date: Oct. 27, 2011

(54) IMPROVED PRODUCTION OF PROTEINS WITH DOWNSTREAM BOX FUSIONS IN PLASTIDS AND IN BACTERIA

(75) Inventors: Maureen R. Hanson, Ithaca, NY (US); Benjamin N. Gray, Somerville, MA (US); Beth A. Ahner, Ithaca, NY (US)

(73) Assignee: CORNELL UNIVERSITY, Ithaca, NY (US)

(21) Appl. No.: 13/059,709

(22) PCT Filed: Aug. 17, 2009
(86) PCT No.: PCT/US2009/053981

§ 371 Date: Feb. 18, 2011

Related U.S. Application Data
(60) Provisional application No. 61/090,808, filed on Aug. 21, 2008.

(51) Int. Cl. A01H 5/00; C07H 21/00; C07K 1/00; C12P 21/00; C12N 15/63; C07H 21/04

(52) U.S. Cl. 800/298; 435/320.1; 536/23.7; 536/23.2; 530/350; 435/69.1; 536/23.1

(57) ABSTRACT

The present invention is directed to the use of certain selected downstream box (“DB”) regions and codon-optimized DB regions to achieve high-level protein expression in transformed organisms or organelles. In particular, high level protein expression in plastids and bacteria can be achieved by fusion of the TetC or NPTII DB region to a gene of interest. Protein expression in a transformed organism or organelle can also be enhanced by optimization of codon usage within the DB region based on codons preferentially found in the DB regions of highly expressed native genes in the organism or organelle. Methods for enhanced protein expression, and related nucleic acid molecules, expression vectors, transformed cells, and transgenic organisms, are provided. The present invention is particularly useful for expressing cellulolytic enzymes, including a suite of several cellulolytic enzymes, in plastids, bacteria and algae.

### STATEMENT REGARDING FEDERALLY SPONSORED RESEARCH OR DEVELOPMENT

This invention was made with Government Support from U.S. Department of Agriculture (under Contract No. USDA NRI 2007-02133) and a U.S National Science Foundation Graduate Research Fellowship. The Government has certain rights in this invention.

### FIELD OF THE INVENTION

This invention relates to genetic engineering of microorganisms and plants for improved expression of a protein of interest. More particularly, the present invention relates to identification of certain selected downstream box (“DB”) regions and to codon modification of any given DB regions to achieve high-level protein expression in transformed organisms or organelles. Methods for enhanced protein expression including in particular, expression of cellulolytic enzymes, and related nucleic acid molecules, expression vectors, transformed cells, as well as transgenic organisms, are provided.

### BACKGROUND OF THE INVENTION

Though cellulosic ethanol is a promising fuel from an environmental standpoint, industrial production and commercialization of cellulosic ethanol has been slow, in large part due to the high cost of cellulases, the enzymes used for enzymatic cellulose hydrolysis. One option for low-cost enzyme production is the use of transgenic plants as a heterologous protein production system. Plant-based protein production can offer economic advantages over more traditional protein production platforms such as bacterial and fungal cultures, especially when the desired protein accumulates to high levels in transgenic plant tissues (e.g., greater than 10% of total soluble protein or “TSP”). In this regard, chloroplast transformation offers an advantage over plant nuclear transformation, as the former technique often results in higher levels of foreign protein accumulation than the latter, improving the economics of production by increasing the protein concentration in harvested plant tissue (Maliga (2003) Trends Biotechnol 21: 20-28). While nuclear transformants typically produce foreign protein up to 1% TSP in transformed leaf tissue, with some exceptional transformants producing protein at 5-10% TSP, chloroplast transformants often accumulate foreign protein at 5-10% TSP in transformed leaves, with exceptional transformants reaching as high as >40% TSP (Maliga (2003)) or greater (Oey et al. (2009), Plant J. 57:436-45).

A major economic advantage of plant-based protein production over one that is microorganism-based is in the scale-up of protein expression. Whereas scale-up of microbial systems requires the purchase and maintenance of large fermentors and associated equipment, scale-up of plant-based protein production only requires the planting of more seed and harvesting of a larger area. Cellulase-expressing transgenic plants may offer significant capital cost savings over more traditional cellulase production via cellulolytic fungi or bacteria.

Cellulases are broadly grouped into two categories, the endoglucanases and exoglucanases, and are grouped into families based on amino acid similarity (Carbohydrate Active Enzymes Database). Endoglucanases act by randomly cleaving cellulose fibers to create glucose oligomers. Exoglucanases processively hydrolyze these glucose oligomers to produce mostly cellobiose. Cellulases have been expressed in plants previously, including from the plastid genome (International Patent WO 98/11235; Yu et al. (2007) J Biotechnol 131: 362-369), but a suite of cellulolytic enzymes has not been expressed in transgenic plants. Efficient enzymatic cellulose hydrolysis requires the concerted action of multiple cellulases with non-redundant activities (Irwin et al. (1993) Biotechnol Bioeng 42: 1002-1013).

The downstream box (or “DB”) region, defined by a short nucleotide sequence immediately downstream of the start codon, has been identified previously as an important regulator of translation efficiency in Escherichia coli (Sprengart et al. (1996) EMBO J 15: 665-674) and in chloroplasts (Kuroda and Maliga (2001a) Nucleic Acids Res 29: 970-975; Kuroda and Maliga (2001b) Plant Physiol 125: 430-436), which use prokaryotic-like translation machinery. The mechanism of translation enhancement by the DB region is unknown (O'Connor et al. (1999) Proc Natl Acad Sci USA 96: 8973-8978), but DB fusions have been used to increase foreign protein accumulation in E. coli (e.g., Keum et al. (2006) Biochem Biophys Res Commun 350: 562-567) and in tobacco chloroplasts. A fusion of the GFP DB to the bacterial EPSPS gene allowed for more than a 30-fold improvement in protein accumulation in tobacco chloroplasts (Ye et al. (2001) Plant J 25: 261-270). However, downstream box fusions do not always result in increased foreign protein accumulation in chloroplasts. For example, silent mutations in the native rbcL and atpB DB sequences decreased NPTII accumulation in chloroplast-transformed tobacco by approximately 35-fold and 2-fold, respectively (Kuroda and Maliga (2001b)). Similarly, a downstream box designed to perfectly base-pair with a region of the ribosomal RNA that was termed the “anti-downstream box” (Sprengart et al. (1996)) resulted in NPTII accumulation over 100-fold lower than that resulting from an NPTII gene lacking this downstream box fusion (Kuroda and Maliga (2001a)).

Identification of DB regions that can predictably enhance foreign protein accumulation for many different proteins in chloroplasts would be of particular importance to the expression of cellulases in transplastomic plants. The identified DB region could be fused to the coding regions of the various cellulases necessary for efficient cellulose degradation (i.e., endoglucanses, exoglucanases, and accessory enzymes) and then inserted into the chloroplast genome of the desired host plant.

### SUMMARY OF THE INVENTION

The present invention is directed to certain downstream box (“DB”) regions and to codon-modified DB regions, and use of such DB regions to achieve high-level protein expression in transformed organisms or organelles.

In one aspect, the present invention provides isolated nucleic acid molecules that include a nucleotide sequence of at least 8-10 contiguous codons of the DB region of the tetC gene or the neo gene.

In one embodiment, the isolated nucleic acid molecules include a nucleotide sequence of at least 8-10 contiguous codons of the DB region of the tetC gene or the neo gene, wherein the DB regions of the tetC gene and the neo gene are set forth in SEQ ID NO: 38 and SEQ ID NO: 40, respectively.

In another embodiment, the isolated nucleic acid molecules contain a nucleotide sequence that encodes a 13 amino acid peptide having a sequence as set forth in SEQ ID NO: 34 or SEQ ID NO: 36.

In another aspect, the present invention provides isolated nucleic acid molecules that include a nucleotide sequence of at least 8-10 codons, which corresponds to a sequence of contiguous codons from the DB region of a gene of interest, except that the nucleotide sequence has been codon-optimized based on the codon usage frequencies of the DB regions of highly expressed native genes in an organism of organelle of choice.

In still another aspect, the present invention provides nucleic acid constructs, i.e., nucleic acid molecules made by genetic engineering techniques, which are useful for high level expression of a protein in plastids, bacteria and algae. The nucleic acid constructs of the present invention contain a nucleic acid molecule described above, i.e., a nucleic acid molecule which includes a DB sequence (either at least 8-10 contiguous codons of the DB region of the tetC gene or the neo gene, or at least a 8-10 codon-optimized segment from the DB region of a gene of interest), wherein such DB sequence-containing nucleic acid is linked immediately downstream of the start codon of a coding sequence.

In one embodiment, the DB sequence-containing nucleic acid in the construct includes 10-15 codons of the DB region of the tetC gene or the neo gene.

In another embodiment, the DB sequence-containing nucleic acid in the construct is heterologous relative to the coding sequence and is codon-optimized.

In still another embodiment, the DB sequence-containing nucleic acid in the construct is native relative to the coding sequence and is codon-optimized.

The coding sequence in operable linkage to the DB sequence-containing nucleic acid can encode any protein of interest, particularly proteins heterologous to the organisms or organelles to be transformed, including and not limited to industrial enzymes (such as a cellulolytic enzyme), and pharmaceutical proteins such as cytokines, antibodies, immunogenic peptides or polypeptides.

In a further aspect, the present invention provides expression vectors and methods for high level expression of proteins in plastids, bacteria and algae by using the DB sequence-containing nucleic acid molecules and constructs described herein.

Transgenic plants, bacteria and algae produced by transformation with the DB sequence-containing nucleic acid molecules and constructs described herein are also provided by the invention.

### BRIEF DESCRIPTION OF THE DRAWINGS

FIGS. 1A-1B. Schematic diagrams of tobacco chloroplast DNA. 1A: Transformed chloroplast DNA, showing the 16S rDNA, trnI, and trnA genes along with the XhoI restriction sites relevant to Southern blot experiments. The T7g10 5′UTR is immediately upstream of the cel6A ORF. NdeI and NheI restriction sites are located at the 5′ end of the DB, and the DB region is fused to the cel6A ORF. The cel6A gene is followed by the psbA 3′UTR (TpsbA) and the aadA expression cassette. The aadA gene is flanked by the psbA promoter and 5′UTR (PpsbA) and the rps16 3′UTR (Trps16). The entire aadA expression cassette is flanked by loxP sites. 1B: Wild-type chloroplast DNA, showing the 16s rDNA, trnI, and trnA genes along with the XhoI restriction sites relevant to Southern blot experiments.

FIG. 2. Southern blot showing XhoI-digested wild-type (WT) and transformed tobacco DNA. A trnA-specific probe hybridized with the expected 3.0 kb band in WT tobacco, and with a 5.7 kb band in transplastomic tobacco.

FIGS. 3A-3D. Immunoblots showing Cel6A protein accumulation in aging leaves. 3A: TetC-Cel6A transformed plants. 3B: NPTII-Cel6A transformed plants. 3C: GFP-Cel6A transformed plants. 3D: Immunoblots were quantified, showing Cel6A accumulation of up to 7.6% TSP in TetCCel6A transformed tobacco leaves, up to 0.9% TSP in NPTII-Cel6A transformed tobacco leaves, and up to 0.3% TSP in GFP-Cel6A transformed tobacco leaves.

FIGS. 4A-4B. Comparison between Cel6A quantification from CMCase activity (gray bars) in tobacco leaves and immunoblot analysis (black bars, from FIG. 3D). 4A: TetC-Cel6A leaf extracts. 4B: NPTII-Cel6A leaf extracts. Leaf protein extracts were used to digest 2% CMC and quantified against a standard curve generated by incubating known amounts of Cel6A with 2% CMC.

FIGS. 5A-5B. Coomassie-stained polyacrylamide gels showing cellulose-affinity purification of TetC-Cel6A. 5A: Tobacco-produced TetC-Cel6A. 5B: E. coli-produced TetC-Cel6A. Crude protein extracts were incubated with cellulose resin, then washed sequentially in Tris (20 mM, pH 7.4) and Tris (20 mM, pH 7.4) with NaCl (0.8 M) buffers. TetC-Cel6A was eluted in ethylene glycol. Ethylene glycol was removed by buffer exchange and eluted TetC-Cel6A was resuspended in Tris (20 mM, pH 7.4).

FIGS. 6A-6C. T1 generation of Cel6A-expressing tobacco. 6A: T1 generation Cel6A-expressing tobacco seedlings planted in MS medium lacking antibiotic were phenotypically indistinguishable from wild-type tobacco. 6B: Immunoblot with protein extracts from aging leaves of GFP-Cel6A, NPTII-Cel6A, and TetC-Cel6A transformed tobacco. 6C: Quantification of the immunoblot in FIG. 6B.

FIG. 7. RNA blotting of cel6A mRNA from T1 generation Cel6A-expressing tobacco. Total RNA was hybridized with a radiolabelled cel6A probe, revealing differences in the accumulation of cel6A transcripts in GFP-Cel6A, NPTII-Cel6A, and TetC-Cel6A tobacco leaves. Major transcripts are seen at 4.0 knt (16s rrn-trnI-cel6A), 3.0 knt (unknown transcript containing both trnI and cel6A), 2.3 knt (trnI-cel6A), and 1.3 knt (cel6A).

FIG. 8A-8D. Accumulation of BglC fused to the TetC, NPTII, and GFP DB regions in the leaves of chloroplast-transformed tobacco. FIGS. 8A-8C show immunoblots for NPTII-BglC, TetC-BglC, and GFP-BglC expression in transformed tobacco chloroplasts that are quantified in FIG. 8D. Fusion of the NPTII DB region to the BglC open reading frame (ORF) resulted in significantly higher accumulation of BglC protein in chloroplasts (8.0-11.6% TSP) than fusion of the TetC (1.6-2.6% TSP) or GFP (<0.3% TSP) DB regions to the BglC ORF.

FIG. 9. E. coli (K12 Strain) Codon Usage Frequencies (black bar: overall CUF; light grey bar: DB CUF).

FIG. 10. N. tabacum chloroplast Codon Usage Frequencies (black bar: overall CUF; light grey bar: DB CUF).

FIGS. 11A-11B. Accumulation of GFP-Cel6A in E. coli BL21(DE3) cells 5 hours after IPTG induction of protein synthesis. FIG. 11A shows an immunoblot that is quantified in FIG. 11B. Codon optimization of the GFP DB region resulted in increased GFP-Cel6A accumulation, with the HF(EcDB)GFP DB region resulting in the highest level of GFP-Cel6A accumulation.

FIGS. 12A-12B. Human IL-2 cDNA and protein sequences. Boxed region in IL-2 protein sequence (SEQ ID NO: 42) (FIG. 12A) is the targeting sequence that was removed for chloroplast expression and corresponds to underlined section of gene sequence (SEQ ID NO: 43) (FIG. 12B). Capital lettered ATG start codon was fused directly to the GCA (alanine) codon following target sequence of native protein to generate the mature protein in transplastomic plants.

FIGS. 13A-13B. Western blot IL-2 tobacco transformants. FIG. 13A shows Western blot analysis of IL-2 tobacco transformants. Antibody used was a mouse monoclonal anti-human IL-2. Lane 1, protein marker; lane 2 and 6, WT control; lane 3 and 7, IL-2 WT transformant 1; lane 4 and 8, IL-2 CM transformant 3-1; lane 5 and 9, IL-2 CM transformant 3-5. (IL-2 CM stands for IL-2 codon modified). FIG. 13B shows an SDS-PAGE picture of protein samples on the Western blot, same order and same amount.

FIG. 14. Western blot IL-2 tobacco transformants (additional plant replicates with standard included). Plant tissue collected from plants was grown in culture box prior to transfer. A total of 50 μg of protein for each sample was loaded to 15% SDS-PAGE gel. Blots were blocked in 5% milk-TTPS buffer overnight. The anti-IL-2 antibody was diluted 1:1000 and hybridized for 4 hours. The secondary antibody was diluted 1:30,000 and hybridized for 1 hour.

### DETAILED DESCRIPTION OF THE INVENTION

The present invention is directed to the use of certain selected or codon-modified downstream box (“DB”) regions to achieve high-level protein expression in transformed organisms or organelles. Specifically, in work leading up to the present invention, it has been determined that production of a heterologous protein in plastids can be enhanced by fusing the downstream box (DB) regions of the TetC and NPTII genes to the N-terminus of the heterologous protein. Further, it has also been identified that production of a heterologous protein in a recipient organism or organelle can be improved by optimizing the codon usage within the DB region of such protein, based on codons preferentially found in the DB regions of highly expressed native genes in the recipient organism or organelle. Accordingly, the present invention provides methods, related nucleic acids, expression vectors, and host cells for high level protein production in plastids, bacteria and algae based on use of certain selected DB regions or codon optimized DB regions. The resulting transgenic plants, bacteria and algae are also provided in the invention.

Downstream Box (DB) Regions Defined

The term “downstream box region” or “DB region”, as used herein, refers to a contiguous nucleic acid segment of at least about 8-10 codons immediately downstream of the start codon of a pertinent gene. Generally speaking, the DB region is not longer than 30 codons, preferably not longer than 20 codons; and more preferably, not longer than 18 codons. In preferred embodiments, the DB region can consist of 10, 11, 12, 13 14, 15 or 16 codons immediately downstream of the start codon.

By “immediately downstream of the start codon” it is meant that the 5′ end of the DB region is within 5 or fewer codons of the start codon; preferably within 4 codons, or more preferably within 3 or 2 codons or even 1 codon from the start codon of a pertinent gene. Alternatively, there are no additional codons between the start codon and the first codon of the DB region.

DB Regions of the TetC and NPTII Genes

In one aspect of the invention, the DB region of the TetC gene (encoding TetC, a non-toxic 47 kD polypeptide fragment of tetanus toxin) or the neo gene (encoding E. coli neomycin phosphotransferase II or “NPTII”) is employed to generate fusion construct with a gene of interest in order to achieve high-level expression in plastids of the protein encoded by the gene of interest.

The full-length tetC (Tregoning et al. (2003) Nucleic Acids Res 31: 1174-1179) and neo (Kuroda and Maliga [2001] Nucleic Acids Res 29: 970-975) genes have been expressed previously from the tobacco plastid genome. However, the present invention provides for the first time the use of the DB regions of TetC and NPTII to drive high-level expression of heterologous proteins in transformed plastids. For example, plastid transformation with a fusion of a 13-codon segment derived from the DB region of TetC to the Thermobifida furca cel6A gene led to an accumulation of enzymatically active Cel6A protein at up to 10.7% of total soluble protein (% TSP) in tobacco leaf, which is at least 5-10 times higher than previously reported attempts of Cel6A expression in plastids (Yu et al. (2007) J Biotechnol 131: 362-369; Ziegelhoffer et al. (1999) Mol Breeding 5: 309-318). As another example, chloroplast-transformed tobacco expressed a fusion protein of a 13 amino acid segment from the DB region of NPTII fused to β-glucosidase (BglC), at 8.0-11.6% TSP, which is a 3.5- to 5-fold increase relative to the levels of β-glucosidase reportedly expressed in tobacco from the nuclear genome (Wei et al. (2004) Plant Biotechnol J 2: 341-350).

In one embodiment, a nucleotide sequence of at least 10 contiguous codons from the DB region of the TetC gene is used in a fusion construct. Preferably, a nucleotide sequence of at least at least 11, 12, 13, 14 or 15 codons from the DB region of the TetC gene is used in a fusion construct. The nucleic acid and amino acid sequences of the first 30 codons of the TetC gene are set forth in SEQ ID NOS: 38 and 39, respectively.

In a preferred embodiment, a nucleotide sequence including at least 10 contiguous codons encoding the N-terminal fragment (“KNLDCWVDNEEDI”, set forth in SEQ ID NO: 34) of TetC is used in a fusion construct. More preferably, the nucleotide sequence includes 11 or 12 contiguous codons, or even more preferably, all 13 codons encoding the N-terminal fragment (SEQ ID NO: 34) of TetC.

In another embodiment, a nucleotide sequence of at least 10 contiguous codons from the DB region of the neo gene is used in a fusion construct. Preferably, a nucleotide sequence of at least at least 11, 12, 13, 14 or 15 codons from the DB region of the neo gene is used in a fusion construct. The nucleic acid and amino acid sequences of the first 30 codons of the neo gene are set forth in SEQ ID NOS: 40 and 41, respectively.

In a preferred embodiment, a nucleotide sequence including at least 10 contiguous codons encoding the N-terminal fragment (“IEQDGLHAGSPAA”, set forth in SEQ ID NO: 35) of NPTII is used in a fusion construct. More preferably, the nucleotide sequence includes 11 or 12 contiguous codons, or even more preferably, all 13 codons encoding the N-terminal fragment (SEQ ID NO: 35) of NPTII.

A DB sequence described above (derived from TetC or NPTII) can be linked to a gene of interest to create a DB fusion construct such that the DB sequence is placed or inserted immediately downstream of the start codon of the gene of interest. As defined above, by “immediately downstream of the start codon” it is meant that the 5′ end of the DB sequence is within 5 or fewer codons of the start codon; preferably within 4 codons, or more preferably within 3 or 2 codons or even 1 codon, from the start codon of the gene of interest.

In some cases, for convenience of cloning, there can be one or more exogenous codons introduced into the junction(s) between the DB sequence and the gene of interest. For example, one or more exogenous codons (i.e., codons heterologous to the gene of interest and the DB sequence) are introduced into the fusion construct 5′ to the first codon of the TetC or NPTII DB sequence or 3′ to the last codon of the DB sequence. As shown in the following examples, an Nhe I site (GCTAGC) was included in the fusion primer design that resulted in the addition of two codons (coding for Ala and Ser) between the start codon of the heterologous gene (cel6A or bglC) and the 5′ end of the DB sequence from TetC, NPTII or GFP. Consequently, the resulting DB fusion proteins, in effect, include an insertion of 15 amino acids between the start codon and the second codon of the heterologous protein. For example, the TetC DB-Cel6A and NPTII DB-Cel6A fusion proteins shown in the following examples include an insertion of 15 amino acids (SEQ ID NO: 36 and SEQ ID NO: 37, respectively) between the start codon and the second codon of the native heterologous protein.

Codon-Optimized DB Regions

In another aspect, the present invention is directed to a method for improved protein production in a recipient organism or organelle, as well as related vectors and host cells for practicing the method, based on use of a DB region that has been optimized in its codon usage to drive the protein expression from a heterologous gene of interest.

In accordance with this aspect of the invention, a DB region is codon optimized based on codons preferentially found in the DB regions of naturally occurring highly expressed genes in the recipient organism or organelle.

Codon optimization has been utilized in order to improve the production of proteins (see, e.g., U.S. Pat. No. 0,292,918 A1, U.S. Pat. No. 5,795,737). However, prior to the present invention, the codon usage has been optimized for the entire coding region of a protein of interest, and optimization is based on the most frequently used codons for the entire length of all known protein coding regions in the genome of the organism or organelle to be transformed. Unique to the present invention, codon optimization is directed only to the DB region of the gene of interest and is based on codons preferentially found in the DB regions of naturally occurring highly expressed genes in the recipient organism or organelle.

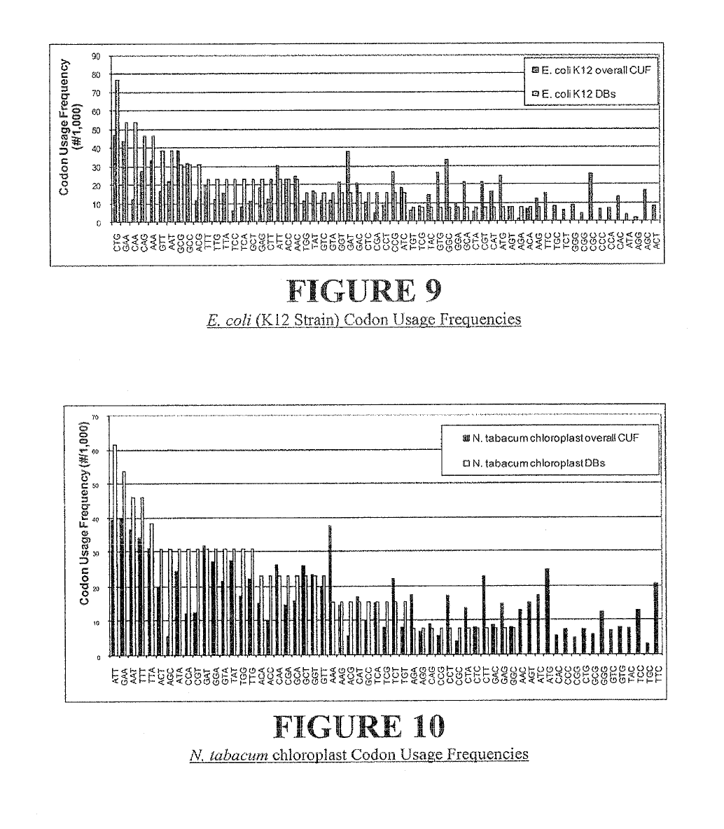

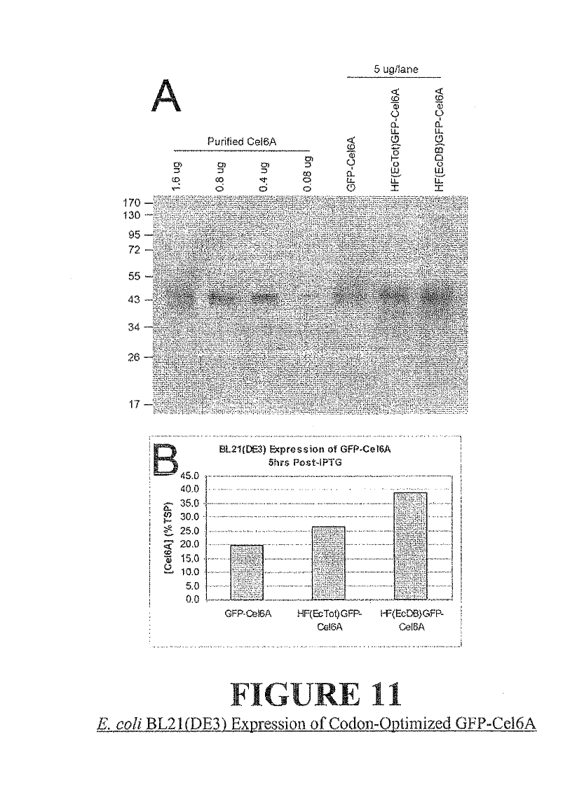

The codon optimization approach provided by the present invention is premised on the unique recognition that the codon usage frequencies in the DB regions can differ from the codon usage frequencies over full-length coding regions, and that specifically altering the codons of only the DB region in the transgene to correspond to the preferred codon usage of the DB regions in the recipient organism or organelle is sufficient to achieve significant improvement in protein expression from the transgene. For example, it has been determined in accordance with the present invention that codon usage frequencies of the 61 amino acid-encoding codons differ between the DB regions of highly expressed native E. coli genes and the entire E. coli (K12) genome (FIG. 9 and Table 1). Similarly, codon usage frequencies (CUFs) of the 61 amino acid-encoding codons for all genes in the N. tabacum chloroplast genome are also different from those for the DB regions of the genes encoding ten highly expressed proteins in the N. tabacum chloroplast genome (FIG. 10 and Table 2). Further, it has been demonstrated herein that alteration of the DB region of the green fluorescent protein (GFP) to include codons preferentially used in the DB regions of highly expressed native E. coli genes allowed for an approximate two-fold improvement in expression of the GFP DB-Cel6A fusion protein from E. coli BL21(DE3) cells (FIG. 11).

According to the present invention, the codon optimization approach provided herein can be used to enhance protein expression in any organism or organelle of choice, including in particular, plants, plant materials (such as plant cuttings, tissues, cells, tissue cultures and seeds), plant organelles (such as, e.g., plastids, including all forms of plastids and not limited to chloroplasts), bacteria (e.g., E. coli), and algae, for example.

In order to optimize a given DB region for protein expression in an organism or organelle of choice, one should first determine the codon usage frequencies of the DB regions of naturally occurring highly expressed genes within such organism or organelle. “Highly expressed genes” refer to genes whose encoded proteins are produced at high levels, i.e., abundant proteins. By “high levels” it is meant proteins accumulating to at least 5% of total soluble protein (5% TSP), preferably at least 7%, 8%, 10%, 12%, or 15% or higher. Information regarding naturally occurring highly expressed genes in many plants, bacteria and algae is generally available through proteomic databases (e.g., the Integr8 Proteome Analysis database) and in scientific literature. Preferably, multiple (e.g., at least a minimum of 8, 9, 10, 15, 20, 25 or more) highly expressed native genes are analyzed to determine the codon usage frequencies of the DB regions of these genes.

Once the codon usage frequencies of the DB regions for an organism or organelle have been determined, a DB region of interest can be altered to employ codons preferentially used in the DB regions of highly expressed native genes. As illustration, Table 1 and Table 2 set forth most preferred (most frequently used), less preferred, and least preferred codons for the DB regions of E. coli and N. tabacum chloroplast. Preferably, after codon optimization, at least 75%, 80%, 85%, 90%, 95%, 98% or 100% of the codons in the DB region of a gene of interest should correspond to the most preferred codons. As demonstrated herein, the DB region of GFP has been codon optimized for E. coli expression such that a most frequently used codon was chosen for each of the 13 amino acids within the DB region of GFP used, i.e., 100% optimization.

In accordance with the present invention, the codon optimization approach described above here, i.e., substitution of synonymous codons preferentially used in the DB regions of genes encoding highly abundant proteins in an organism or organelle, can be applied to any given DB region to improve protein accumulation in the organism or organelle. This includes, for example, a DB region fused to a heterologous gene of interest (i.e., the DB region is non-native relative to the gene of interest) as in the case of fusion of the GFP DB region to the cel6A gene; as well as the native DB region of any gene of interest that is heterologous to the recipient organism or organelle. Compared to conventional codon optimization of an entire coding region, the approach provided by the present invention is much more convenient yet effective.

Gene of Interest

The DB region of the tetC or the neo gene, as described above, and any DB region that has been codon-optimized in accordance with the present invention, can be employed to drive high-level protein expression from a gene of interest. Genes of interest contemplated by the present invention include, but are not limited to, genes encoding industrial enzymes, pharmaceutical proteins such as cytokines, antibodies, immunogenic peptides or polypeptides.

In one embodiment, the gene of interest encodes a cellulolytic enzyme. The term “cellulolytic enzyme”, as used herein, refers to a cellulose-degrading enzyme, which includes cellulases, cellobiohydrolases, cellobioses and other enzymes involved in breaking down cellulose and hemicelluloses into simple sugars such as glucose and xylose. Generally speaking, at least three different enzymatic activities are required to effectively reduce cellulose to cellobiose and then to glucose: β-1,4-endoglucanases (also called endocellulases), which cleave β-1,4-glycosidic linkages randomly along the cellulose chain; β-1,4-exoglucanases (also called cellobiohydrolases or exocellulases) that cleave cellobiose from either the reducing or the non-reducing end of a cellulose chain; and 1,4-β-D-glucosidases (also called cellobiosases) that hydrolyze aryl- and alkyl-β-D-glucosidases.

In preferred embodiments, a cellulolytic enzyme is of a microbial origin, e.g., a bacterial or fungal original. For example, cellulolytic enzymes from a bacterium of the genus of Thermobifida (e.g., T. fusca), a bacterium of B. subtilis, bacteria of the genus Clostridium (e.g., C. thermocellum), bacteria of the genus Acidothermus (e.g., A. cellulolyticus) or a fungus (e.g., Aspergillus niger or Trichoderma reesei), are all suitable for expression using a DB region in accordance with the present invention. Genes encoding numerous microbial cellulolytic enzymes have been documented in the art and are available through, e.g., GenBank or the Carbohydryate Active Enzymes (CAzy) database.

In a specific embodiment, the gene of interest is a bacterial gene encoding a cellulase, either an endocellulase or an exocellulase. An example of a cellulase gene is the Thermobifida fusca cel6A gene, the nucleotide and amino acid sequences of which are set forth in Lao et al (1991) J Bacterial 173: 3397-3407 and in GenBank entry AAC06388.1, respectively. Plastid transformation with a fusion of TetC DB-cel6A led to an accumulation of enzymatically active Cel6A protein in tobacco leaf, at a level of at least 5-10 times higher than previously reported attempts of Cel6A expression in plastids.

In another specific embodiment, the gene of interest is a β-glucosidase gene. An example of a β-glucosidase gene is the T. furca bglC gene (nucleotide and amino acid sequences in GenBank entries AF086819.2 and AAZ54975.1, respectively). Another example is the A. niger Bgl1 gene (Wei et al. (2004)). The present invention is believed to have provided for the first time β-glucosidase expression from a plastid genome. Chloroplast-transformed tobacco expresses an NPTII DB-BglC fusion at a level with a 3.5- to 5-fold increase relative to the levels of β-glucosidase production from nuclear genome of tobacco previously reported by Wei et al. (2004).

In still another embodiment, several genes of interest encoding a suite of cellulolytic enzymes are expressed simultaneously in plastids, each being driven by a selected or codon-optimized DB region described hereinabove. A complex suite of cellulolytic enzymes is often required for efficient cellulose hydrolysis. By “a suite of cellulolytic enzymes” it is meant herein a combination of at least one endocellulase, one exocellulase and one β-glucosidase.

In a further embodiment, the gene of interest encodes a pharmaceutical or therapeutic protein such as a cytokine (e.g., interleukin-2).

Expression Vectors

A transgene construct, which includes a selected DB region or a codon-optimized DB region linked to a coding sequence of interest, is generally placed in a vector which is then transformed into a desirable recipient cell or organism for expression.

The transgene can be placed in the vector in an operable and direct linkage to a promoter, which will direct its expression in the target cell or organism. A number of promoters are well known to be suitable for controlling expression of chloroplast genes; for example, the psbA, 16S rRNA, atpB, or rbcL promoter (Maliga (2002) Current Opinion in Plant Biology 5:164-172; U.S. Pat. No. 5,877,402 to Maliga et al. and U.S. Pat. No. 6,987,215 to Maliga et al.). Promoters suitable for use in directing expression in bacteria are also well known and include, e.g., the lac promoter or the T7 promoter. Alternatively, it is not necessary for the transgene to have its own promoter. The expression of a transgene in plastids can be achieved based on read-through transcription from the promoter of an upstream gene, resulting in a polycistronic message, as shown in examples herein below.

The transgene typically is also operably linked to a 3′ untranslated region from a chloroplast gene, in order to provide transcription termination and/or message stability. A number of such 3′ regions are well known, for example, the 3′ region from rbcL or psbA (see, e.g., Maliga (2002), supra, U.S. Pat. No. 5,877,402 and U.S. Pat. No. 6,987,215).

In order to provide a means of selecting the desired transformant, the vectors typically contain a selectable marker gene such that cells containing such a gene will have a distinctive phenotype for purposes of identification. Examples of selectable marker genes include genes encoding polypeptides which confer resistance to a selective substance, e.g., antibiotic (such as the bacterial aadA gene which confers resistance to spectinomycin and streptomycin in plant cells), kanamycin, plant herbicides (such as phosphinothricin), and inhibitors such as indole analogue 4-methylindole (4MI) or the tryptophan analogue 7-methyl-DL-tryptophan (7MT).

For transformation and expression in plastids, the vector carrying a transgene preferably includes nucleic acid sequences that bear homology to the target genome and can mediate integration of the pertinent portion of the vector (including the transgene and a selectable marker gene, for example) into the genome (plastid genome). For example, a transgene together with a selectable marker can be flanked by nucleic acid sequences which bear homology to a target site of the plastid genome of a plant species and mediate integration of the transgene into the plastid genome by homologous recombination (a double crossover event). Various plant plastid genome sequences, as well as vectors containing convenient homologous sequences for transformation, have been documented in the art and are available for use in practicing the present invention.

The vectors that carry a transgene of interest can also contain additional sequences for enhancement or regulation of expression, and intracellular targeting or localization. In addition, the vectors can include sequences (such as linkers, restriction endonuclease sites, origin of replication, for example) which allow for amplification, modification or manipulation of the vector. In certain embodiments, the vectors are capable of replication and propagation to a relatively high copy number in E. coli.

Transformation

Methods of bacterial transformation have been well described in the art, including transformation based on chemicals (such as calcium phosphate, DEAE-dextran or others), liposome fusion and viral/phage infection, and mechanical or electrical means such as microinjection, electroporation, particle bombardment (gene gun) and sonoporation.

For transformation of plant plastids, several methods have been described, e.g., in U.S. Pat. No. 5,877,402 to Maliga et al., which include, but are not limited to, polyethylene glycol (PEG) treatment of protoplasts, bombardment of cells or tissues with microprojectiles coated with the transforming DNA (also referred to as “biolistic DNA delivery”) and temporary holes cut by a UV laser microbeam. Other methods include, calcium phosphate treatment of protoplasts, electroporation of isolated protoplasts, femtosyringe injection of chloroplasts, and agitation of cell suspensions with microbeads coated with the transforming DNA. Alternatively, plastid transformation may be achieved by polyethylene glycol (PEG) treatment of protoplasts in the presence of the transforming DNA. Methods for stable plastid transformation in PEG-treated tobacco protoplasts are described by Golds et al., Bio/Technology, 11: 95-97 (January, 1993).

The biolistic method described in U.S. Pat. No. 5,877,402 to Maliga et al. is a preferred method for practice of the present invention. The method described therein for tobacco can be readily adapted to other plant species. Generally, the organelle is hit by a DNA-coated tungsten or gold particle carrying multiple copies of the vector containing the transforming DNA. Two days after bombardment, the bombarded tissue is transferred into selective media and selection pressure is maintained throughout cellular proliferation in order to obtain a homoplasmic organelle and finally a homoplasmic cell, which can take at least 16 to 17 cell divisions. Shoots are then subcultured on the same selective media to ensure production and selection of homoplasmic shoots.

Transgenic Plants and Algae

Organisms transformed with a transgene, particularly transgenic plants, constitute another embodiment of the present invention. Plants containing chloroplast transgenes have been produced in a number of species, including tobacco, tomato, Chlamydomonas, sugar beet, poplar, carrot, lettuce, rice, and members of the Brassica genus.

In a specific embodiment, the transgenic plant is transgenic tobacco.

In another embodiment, the transgenic plant is transgenic tobacco which expresses one or more cellulolytic enzymes at high levels. For example, tobacco that expresses TetC DB-Cel6A, and tobacco that expresses NPTII DB-BglC, are specific transgenic plants provided by the present invention and represent novel sources of low-cost cellulolytic enzymes.

In another embodiment, the invention provides transgenic algae having a transgene incorporated in its chloroplast genome. Algae having chloroplast transgenes have been generated and reported in the literature (see, e.g., Lapidot et al., Plant Physiology (2002) 129: 7-12).

### Example-1

In this Example, the Thermobifida fusca cel6A gene encoding an endoglucanase was fused to three different downstream box (DB) regions (from GFP, TetC and NPTII) to generate cel6A genes with 13 amino acid fusions at the N-terminus of the encoded protein. The DB-Cel6A fusions were inserted into the tobacco (Nicotiana tabacum cv. Samsun) chloroplast genome for protein expression. Accumulation of Cel6A protein in transformed tobacco leaves varied over approximately two orders of magnitude, dependent on the identity of the DB region fused to the cel6A open reading frame (ORF). Additionally, the DB region fused to the cel6A ORF affected the accumulation of Cel6A protein in aging leaves, with the most effective DB regions allowing for high level accumulation of Cel6A protein in young, mature, and old leaves, while Cel6A protein accumulation decreased with leaf age when less effective DB regions were fused to the cel6A ORF. In the most highly expressed DB-Cel6A construct, enzymatically active Cel6A protein accumulated at up to 10.7% of total soluble leaf protein (% TSP).

The highest accumulation of TetC-Cel6A observed in this Example, 10.7% TSP, is five to ten times higher than the Cel6A accumulation reported previously from chloroplasts transformed to express Cel6A behind the rbcL DB region (Yu et al., 2007). Further, TetC-Cel6A protein remained at a high concentration in older leaves of transformed tobacco, in contrast with chloroplast expression of rbcL-Cel6A (Yu et al., 2007). A report of nuclear Cel6A expression showed accumulation at only 0.1% TSP (Ziegelhoffer et al. (1999) Mol Breeding 5: 309-318). Chloroplast expression of the TetC-Cel6A protein has therefore improved the accumulation of Cel6A protein over 100-fold and allowed for the accumulation of active enzyme in aging leaves.

Materials and Methods

Cloning and Plasmid Construction

Tobacco plastid DNA containing the trnI (tRNA-Ile) and trnA (tRNA-Ala) genes (nt 104500-106205 in Genbank entry Z00044) was PCR-amplified using primers ptDNA-fwd and ptDNA-rev, adding a SmaI site at the 5′ end of this DNA and amplifying a HindIII site from the native plastid DNA sequence. This PCR product was SmaI-HindIII digested and ligated into a pUC19 backbone to generate plasmid pPTDNA. Primers lox-PpsbA-fwd and PpsbA-aadA-rev were used to amplify the psbA promoter (PpsbA; nt 1610-1834 in Genbank entry Z00044) from tobacco plastid DNA and to add NsiI and PstI sites and a loxP recombination site to the 5′ end. Primers PpsbA-aadA-fwd and aadA-Trps16-rev were used to amplify the aadA gene from plasmid pCT08 (Shikanai et al. (2001) Plant Cell Physiol 42: 264-273), and these two PCR products were combined by overlap extension PCR to generate a PpsbA-aadA fragment. Primers aadA-Trps16-fwd and Trps16-lox-rev were used to amplify the rps16 terminator (Trps16) from tobacco plastid DNA (nt 4938-5096 in Genbank entry Z00044), adding a loxP recombination site and an NsiI site at the 3′ end. Overlap extension PCR was used to add Trps16 to the PpsbA-aadA fragment generated above. The aadA cassette generated by this overlap extension PCR was digested by NsiI and ligated into NsiI-linearized pPTDNA to generate pPTDNA-aadA. An NdeI site was removed from the pUC19 backbone in pPTDNAaadA using primers rmvNdeI1, rmvNdeI2, rmvNdeI3, and rmvNdeI4. PCR product rmvNdeI1rmvNdeI4 was digested by AatII and ApaI and ligated into a pPTDNA-aadA backbone generated by AatII-ApaI digestion. The resulting plasmid was pPTDNA-aadA-NdeIdel.

Primers T7-fwd and T7-rev were used to amplify the T7g10 5′UTR (Kuroda and Maliga 2001a) from plasmid pNS6 (Spiridonov and Wilson (2001) Curr Microbial 42: 295-301), adding PstI and AscI sites to the 5′ end and an NheI site to the 3′ end. Primers GFPCel6A-fwd and Cel6A-TpsbA-rev were used to amplify the T. fusca cel6A gene lacking its signal peptide from pGG86 (Ghangas and Wilson (1988) Appl Environ Microb 54: 2521-2526), adding an NheI site and the first thirteen amino acids from green fluorescent protein (GFP) (Ye et al., 2001) immediately downstream of the start codon and a NotI site immediately downstream of the cel6A stop codon. The psbA 3′UTR (TpsbA; nt 443-536 in Genbank entry Z00044) was amplified from tobacco plastid DNA using primers Cel6A-TpsbA-fwd and TpsbArev, introducing a Nod site at the 5′ end of TpsbA and a PstI site at the 3′ end of TpsbA. The GFPCel6A-fwd/Cel6A-TpsbA-rev and Cel6A-TpsbA-fwd/TpsbA-rev PCR products were combined by overlap extension PCR using primers GFPCel6A-fwd and TpsbA-rev. This overlap extension PCR product was NheI digested and ligated to the NheI-digested T7-fwd/T7rev PCR product. The resulting Cel6A cassette containing the cel6A gene flanked by the T7g10 5′UTR and TpsbA was PstI digested and ligated into PstI-linearized pPTDNA-aadA-NdeIdel, resulting in plasmid pGFPCel6A. Plasmid pGFPCel6A was used as a template for amplification of cel6A genes containing 13-amino acid fusions from the neo gene (Kuroda and Maliga 2001a) and from the TetC gene (Tregoning et al., 2003) using primers NPTIICel6A-fwd/Cel6ATpsbA-rev and TetCCel6A-fwd/Cel6A-TpsbA-rev, respectively. The resulting PCR products were NheI/NotI digested and ligated into the NheI/NotI backbone of pGFPCel6A to generate pNPTIICel6A and pTetCCel6A, respectively. All plasmids were maintained in NEB-5-alpha E. coli (New England Biolabs, Ipswich, Mass.).

Plasmids pGFPCel6A, pNPTIICel6A, and pTetCCel6A were NheI-NotI digested and the resulting cel6A fragments were gel purified. The cel6A fragments were ligated into the NheI-NotI backbone of pNS6 (Spiridonov and Wilson 2001) to generate plasmids pGFPCel6AEC, pNPTIICel6AEC, and pTetCCel6AEC, respectively. These plasmids were maintained in NEB5-alpha E. coli (New England Biolabs, Ipswich, Mass.) and were also transformed into BL21(DE3) E. coli cells (Invitrogen, Carlsbad, Calif.) for protein production.

Chloroplast Transformation

Tobacco chloroplasts were transformed by the particle bombardment method (Svab and Maliga 1993). Briefly, plasmid DNA was coated onto 0.6 micron gold beads (Bio-Rad, Hercules, Calif.). Two-week old tobacco seedlings (Nicotiana tabacum cv. Samsun) were bombarded with the DNA-coated beads. Leaves from bombarded seedlings were cultured on RMOP medium containing 500 mg/L spectinomycin (Svab and Maliga (1993) P Natl Acad Sci USA 90: 913-917). Newly generated shoots were screened via PCR for insertion of the cel6A gene at the anticipated site in the chloroplast genome, and positive transformants were transferred to MS medium containing 500 mg/L spectinomycin for rooting. Leaves from rooted plants were subjected to further rounds of tissue culture on RMOP with spectinomycin to obtain homoplasmic transformants. Homoplasmic transformants were transferred to pots and grown in a greenhouse to produce seed.

Southern Blotting

Leaf samples were flash frozen in liquid nitrogen, then finely ground in Eppendorf tubes. 2×CTAB buffer (2% hexadecyltrimethyl ammonium bromide, 1.4 M sodium chloride, 20 mM EDTA, 100 mM Tris pH 8.0, 0.2% β-mercaptoethanol) was added to the ground leaf samples and incubated for one hour at 65° C. DNA was extracted by two sequential phenol extractions followed by isopropanol precipitation. The isopropanol pellet was resuspended in TE buffer (10 mM Tris pH 8.0, 1 mM EDTA) and treated with RNase A (Invitrogen, Carlsbad, Calif.) for one hour at 37° C. DNA was isolated and RNase removed from this solution by phenol extraction. The aqueous phase of this phenol extraction was ethanol precipitated to isolate DNA, which was resuspended in H₂O.

Isolated DNA was completely digested by XhoI and then electrophoresed in 1% agarose. DNA was transferred from the agarose gel to a Hybond N+ membrane (Amersham Biosciences, Piscataway, N.J.). Primers probe-fwd and ptDNA-rev were used to PCR amplify a portion of the trnA gene from wild-type tobacco DNA. This PCR product was used to synthesize a ³²P-labeled probe using the Ambion DECAprime II Random Primed DNA Labeling Kit (Ambion, Austin, Tex.) according to manufacturer's instructions. The ³²P-labeled probe was hybridized with the membrane, washed, and visualized using a Phosphorimager screen (Molecular Dynamics, Sunnyvale, Calif.).

SDS-PAGE and Immunoblotting

Tobacco leaf samples were frozen in liquid nitrogen and then finely ground in eppendorf tubes. Protein extraction buffer (20 mM Tris, pH 7.4, 1% Triton X-100, 0.1% SDS, 1 mM PMSF, 0.01% β-mercaptoethanol) was added to ground leaf samples and vortexed. Supernatant was recovered following a five-minute centrifugation at 16,000×g. The concentration of the protein contained in the supernatant was determined from a bovine serum albumin calibration curve using the Bio-Rad Protein Assay (Bio-Rad, Hercules, Calif.).

Protein samples were electrophoresed in 12% polyacrylamide gels, then transferred to nitrocellulose membranes (Pierce, Rockford, Ill.). Membranes were blocked by incubation with 5% milk in TBST (100 mM Tris, pH 7.6, 685 mM sodium chloride, 0.5% Tween-20), then incubated with anti-Cel6A antibody (kindly provided by David Wilson, Cornell University, Ithaca, N.Y.) diluted 1:100,000 in 5% milk in TBST. Secondary antibody was horseradish peroxidase-conjugated anti-rabbit polyclonal antibody (Sigma, St. Louis, Mo.) diluted 1:25,000 in 5% milk in TBST. Membranes were incubated with SuperSignal West Dura Extended Duration Substrate (Pierce, Rockford, Ill.) and visualized on CL-Xposure film (Pierce, Rockford, Ill.). Purified Cel6A protein for quantitation was kindly provided by David Wilson (Cornell University, Ithaca, N.Y.). Blots were quantified using Scion Image software (Scion Corporation, Frederick, Md.).

Cel6A Production in E. coli

BL21(DE3) cells containing the pGFPCel6AEC, pNPTIICel6AEC, or pTetCCel6AEC plasmid (described above) were grown in LB medium containing kanamycin and Cel6A protein expression was induced with 0.1 mM IPTG. Induced cells were harvested by centrifugation and the spent cell culture medium was removed. Cells were resuspended in Tris (100 mM, pH 7.4) supplemented with 1 mM PMSF, then lysed in Tris (100 mM, pH 7.4) plus 1% SDS and 0.1% β-mercaptoethanol.

Cel6A Purification and N-Terminal Sequencing

TetC-Cel6A protein was purified from tobacco leaf crude protein extract. Crude protein was extracted as described above from tobacco leaves transformed with pTetCCel6A. The crude leaf protein extract was incubated with CBind 200 cellulose resin (Invitrogen, Carlsbad, Calif.) and mixed to allow TetC-Cel6A protein to bind the cellulose. After Cel6A was allowed to bind the resin, the supernatant was removed. The cellulose resin was washed once with Tris (20 mM, pH 7.4), then washed twice with (20 mM, pH 7.4) plus 0.8 M NaCl. TetC-Cel6A was eluted in ethylene glycol. Buffer exchange and protein concentration was performed using a MacroSep column (30,000 MWCO; Pall, East Hills, N.Y.), and purified TetC-Cel6A was re-suspended in Tris (20 mM, pH 7.4). Purity of the eluted TetC-Cel6A was assessed by Coomassie staining a 12% polyacrylamide gel.

GFP-Cel6A, NPTII-Cel6A, and TetC-Cel6A proteins were purified from the appropriate BL21(DE3) E. coli cell protein extract essentially as described above for purification of chloroplast-produced TetC-Cel6A, except that the resin was loaded into a chromatography column.

For N-terminal sequencing, eluted TetC-Cel6A was electrophoresed in a 12% polyacrylamide gel and transferred to a nitrocellulose membrane as described above. The nitrocellulose membrane was Ponceau stained and the TetC-Cel6A bands were excised from the membrane for sequencing. N-terminal sequencing of tobacco- and E. coli-produced TetC-Cel6A was performed at the Penn State University Core Facility (Hershey, Pa.).

Enzyme Activity Assays

Crude leaf protein extracts from T0 tobacco transformants were used to assess Cel6A enzyme activity against carboxymethyl cellulose (CMC). Two different amounts of total protein were added to 2% (w/v) CMC in Hepes buffer (50 mM, pH 7.0): 5 and 2.5 μg leaf protein extract from a TetC-Cel6A expressing plant, and 10 and 5 μg leaf protein extract from an NPTII-Cel6A expressing plant. Eighty microliter reactions were carried out in eppendorf tubes for sixteen hours at 50° C. while mixing. A blank control containing Hepes buffer with no CMC was included to account for any sugar present in the crude protein extract. Reducing sugar content was measured in 96-well plates using a DNS assay protocol adapted from Ghose (1987, Pure Appl Chem 59: 257-268). A standard curve for quantification of Cel6A concentration in the crude protein extracts was generated by measuring reducing sugar release by known amounts of purified Cel6A protein added to a wild-type tobacco protein extract and incubated with 2% CMC.

RNA Blotting

T1 seeds were collected from self-pollinated T0 transformants. The seeds were planted in soil and transferred to individual pots in a greenhouse. Ninety-three days after planting, when the tobacco plants each had approximately 30 leaves, leaf samples were taken from young, mid-, and old leaves (i.e., approximate leaf numbers 28, 15, and 2, respectively) and frozen in liquid nitrogen for protein and RNA extraction. RNA was extracted from leaf samples using Trizol (Invitrogen, Carlsbad, Calif.) according to the manufacturer's instructions. RNA concentration was quantified based on spectrophotometric absorption at 260 nm. Three micrograms of total RNA were loaded in a 1% agarose gel for electrophoresis. Following electrophoresis, RNA was transferred to a Hybond N+ membrane (Amersham Biosciences, Piscataway, N.J.). RNA was detected by hybridization with ³²P-labeled probes. PCR probes were labeled using the DECAprime II Random Primed DNA Labeling Kit (Ambion, Austin, Tex.). Primer pairs for the PCR probes used for RNA detection were Iprobe-fwd/Iprobe-rev (trnI) and C6probe-fwd/Cel6ATpsbA-rev (cel6A). Following hybridization with radiolabelled probes, the membrane was exposed to a Phosphorimager screen (Molecular Dynamics, Sunnyvale, Calif.) for detection. Radiolabel was removed from the membrane by exposure to a boiling solution of 0.1% (w/v) SDS between each hybridization.

Results

Chloroplast Transformation

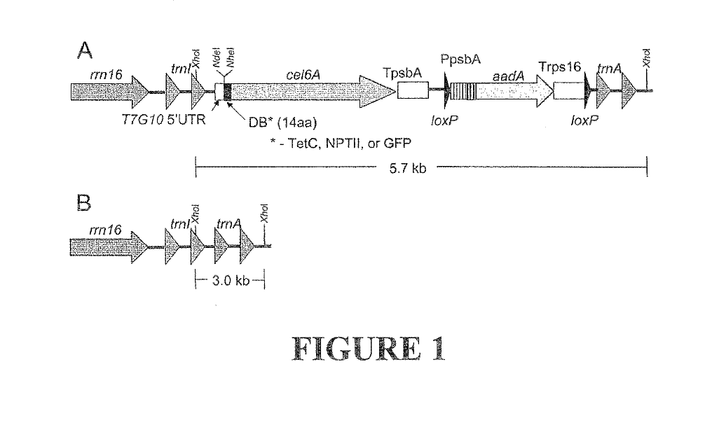

Tobacco (N. tabacum cv. Samsun) chloroplast transformants were generated via particle bombardment using three plasmids containing the elements diagrammed schematically in FIG. 1A. Each plasmid vector contained a gene coding for the mature Cel6A protein (with the native signal peptide removed) with an NheI site immediately downstream from the start codon, followed by the first 13 codons from TetC, NPTII, or GFP, respectively. The cel6A constructs are promoterless, relying on read-through transcription from the upstream Prrn promoter. The aadA gene is placed behind the psbA promoter to ensure high-level expression of aadA for antibiotic resistance. The aadA cassette, containing the psbA promoter, the aadA ORF, and the psbA 5′UTR and rps16 3′UTR, is flanked by loxP sites for future cre-mediated marker gene removal (Corneille et al. (2001) Plant J 27: 171-178).

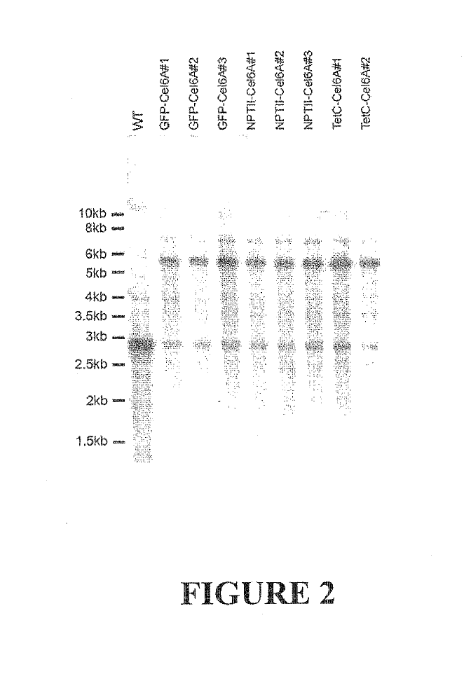

Chloroplast transformants derived from the vectors diagrammed in FIG. 1A were identified via PCR using primers trnIint-fwd and Cel6Aint-rev. Following several rounds of tissue culture regeneration, DNA was isolated from transplastomic plants and digested with XhoI. The schematic diagrams in FIGS. 1A and 1B show the locations of the relevant XhoI sites in transformed and wild-type tobacco chloroplasts, respectively, one internal to the trnI gene and the other downstream of the trnA gene. Homoplasmic plants were confirmed by Southern blotting, shown in FIG. 2. Wild-type tobacco showed a band at the expected size of 3.0 kb, while a trnA probe hybridized with a 5.7 kb band in transformed plants. Faint 3.0 kb bands in transformed plants are assumed to result from the transfer of chloroplast DNA to the nucleus (Ruf et al. (2000) J Cell Biol 149: 369-378). Bands at approximately 7 kb and 2.5 kb are assumed to result from unintended recombination events within transformed chloroplasts and represent a minor fraction of the chloroplast DNA. Homoplasmic plants were transferred to soil and grown in greenhouse conditions to collect seed. Each lane in FIG. 2 represents a homoplasmic plant derived from a unique transformation event.

Protein Accumulation in T0 Transplastomic Transformants

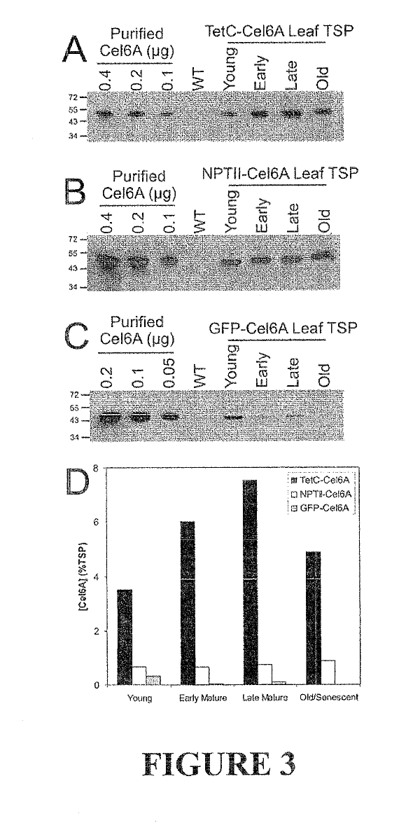

Protein was extracted from the leaves of homoplasmic tobacco transformants for immunoblotting. Cel6A protein accumulation in young leaves of homoplasmic plants transformed with a given construct (i.e., pTetCCel6A, pNPTIICel6A, or pGFPCel6A) was consistent among plants derived from independent transformation events. One plant transformed with each construct was therefore selected for further characterization. FIGS. 3A-3C show that TetC-Cel6A accumulated to significantly higher levels than NPTII-Cel6A, which in turn accumulated to significantly higher levels than GFP-Cel6A. Additionally, Cel6A protein concentration varied with leaf age. TetC-Cel6A protein concentration increased from approximately 3.5% TSP to 7.6% TSP as leaves aged, then decreased in the oldest leaves assayed. NPTII-Cel6A protein concentration remained steady at approximately 0.7-0.9% TSP through plant development. GFP-Cel6A protein accumulated to approximately 0.3% TSP in young leaves, then dropped off quickly as leaves aged to levels that were below detection limits in the oldest leaves.

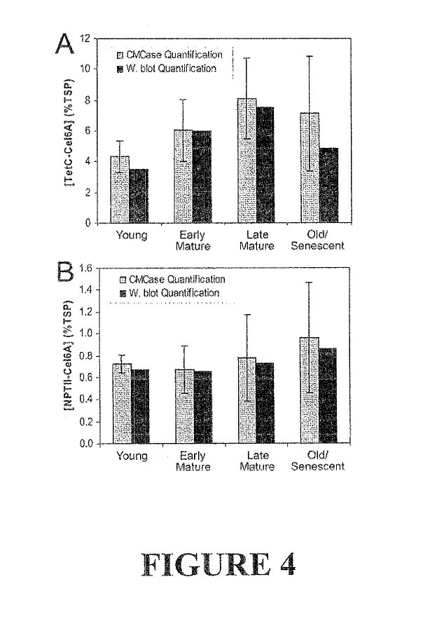

Activity assays using carboxymethylcellulose (CMC) as a substrate were also used to quantify Cel6A accumulation in aging leaves of T0 transplastomic plants. FIGS. 4A-4B show the results of these CMCase activity assays. Quantification of CMCase activity in T0 leaf protein extracts was in good agreement with immunoblot-based quantification of Cel6A protein accumulation, with no statistically significant difference in Cel6A accumulation as calculated by these two methods. This demonstrates that all or nearly all of the Cel6A protein produced in tobacco chloroplasts was active against CMC. The CMCase activity assay using protein extracted from transformed GFP-Cel6A-expressing tobacco was inconclusive, owing to the relatively low expression of GFP-Cel6A in these plants; error associated with quantification of CMCase activity (approximately ±0.5% TSP) is significantly larger than the accumulation of GFP-Cel6A (approximately 0.3% TSP), making the interpretation of tobacco chloroplast-produced GFP-Cel6A CMCase activity difficult. CMCase activity assays using purified Cel6A lacking any DB fusion, TetC-Cel6A, NPTII-Cel6A, and GFP-Cel6A indicated that CMCase activity was similar among these enzymes (data not shown). This indicates that the DB fusions to the Cel6A protein used here do not affect enzyme function.

TetC-Cel6A Purification and N-Terminal Sequencing

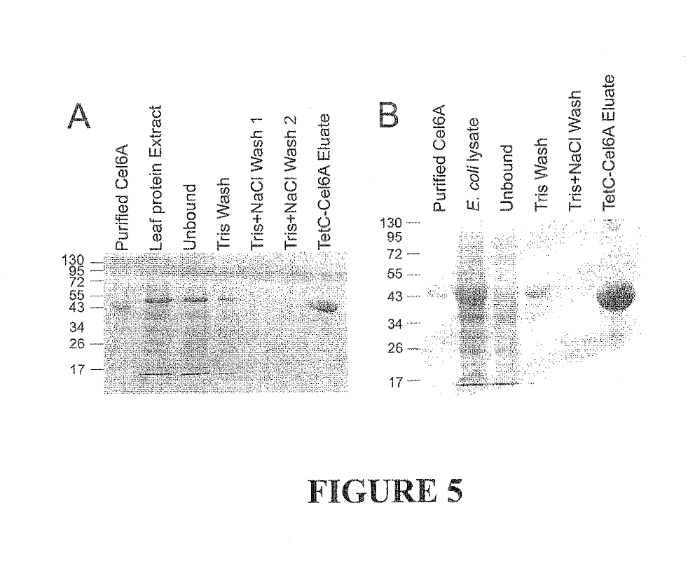

Tobacco chloroplast- and BL21(DE3) E. coli-produced TetC-Cel6A were purified to homogeneity from crude protein extracts by cellulose affinity purification. FIGS. 5A and 5B show Coomassie stained polyacrylamide gels with purified TetC-Cel6A from tobacco chloroplasts and from E. coli, respectively. This figure shows one-step purification of TetCCel6A from both protein expression platforms with very little contamination.

The five N-terminal amino acids of chloroplast- and E. coli-produced TetC-Cel6A were sequenced by Edman degradation. It was determined that f-Met was cleaved from both chloroplast- and E. coli-produced TetC-Cel6A, resulting in an N-terminal alanine residue.

Characterization of Protein Accumulation in T1 Generation Cel6A-Expressing Tobacco

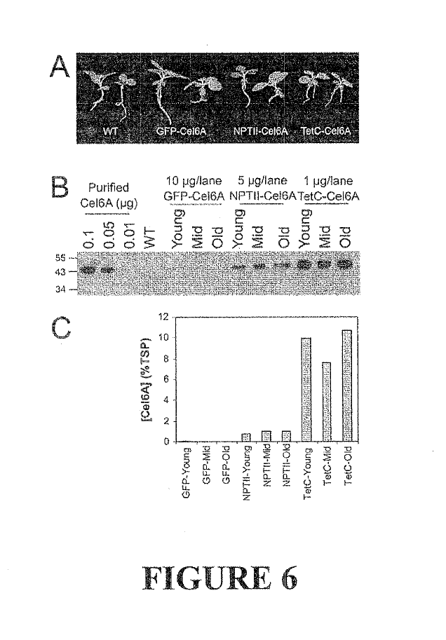

Seed was collected from homoplasmic T0 transformants identified in FIG. 2 and was planted in MS medium lacking antibiotic. FIG. 6A shows WT, GFP-Cel6A, NPTII-Cel6A, and TetC-Cel6A seedlings grown from seed in MS medium. Cel6A-expressing plants were phenotypically indistinguishable from WT tobacco. No growth defects were observed in any of the Cel6A-expressing tobacco lines throughout the life cycle from germination to seed production.

Seed was also planted in soil and T1 generation plants were grown in greenhouse conditions to analyze Cel6A accumulation in aging leaves of T1 plants. FIG. 6B shows the results of an immunoblot with protein extracted from aging leaves of T1 plants. Quantification of this immunoblot in FIG. 6C shows that Cel6A protein accumulation in T1 plants agreed qualitatively with the protein accumulation in T0 plants, with TetC-Cel6A accumulating to higher levels than NPTII-Cel6A, which in turn accumulated to higher levels than GFP-Cel6A. In T1 plants, TetC-Cel6A accumulated to 7.6-10.7% TSP, NPTII-Cel6A accumulated to 0.8-1.0% TSP, and GFP-Cel6A accumulated to ≦0.1% TSP. Protein accumulation in the T1 TetC-Cel6A plant tested showed less variation with leaf age than in the T0 plant (FIGS. 3A, 3D, and 4A). GFP-Cel6A accumulation decreased with leaf age in the T1 plant tested, in agreement with GFP-Cel6A accumulation in the T0 plant tested (FIGS. 3C and 3D).

Characterization of cel6A mRNA in T1 Generation Cel6A-Expressing Tobacco

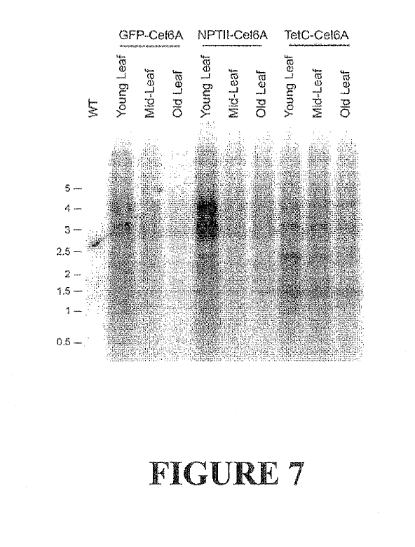

In order to determine whether the differences in Cel6A protein levels were reflected by RNA-level expression of the transgene, levels of cel6A mRNA were examined by probing RNA blots. FIG. 7 shows an autoradiogram of a blot that used RNA extracted from the same leaves used for the immunoblot in FIG. 6B. FIG. 7 shows that monocistronic cel6A transcript (at 1.3 knt) was most abundant in TetC-Cel6A tobacco. Monocistronic cel6A transcript was least abundant in GFP-Cel6A transformed tobacco, with NPTII-Cel6A plants showing an intermediate level of monocistronic cel6A transcript. Dicistronic trnI-cel6A transcript (at 2.3 knt) was also most abundant in TetC-Cel6A tobacco. Tricistronic 16s rrn-trnI-cel6A and an incompletely characterized transcript containing both cel6A and trnI (at 4.0 knt and approximately 3.0 knt, respectively) accumulated to approximately equal levels in TetC-Cel6A, NPTII-Cel6A, and GFP-Cel6A plants. RNA bands were identified on the basis of predicted transcript sizes and were confirmed on RNA blots with a trnI-specific probe.

### Example-2

This Example describes chloroplast production of Beta-glucosidase (BglC from T. fusca) using three DB fusions in tobacco.

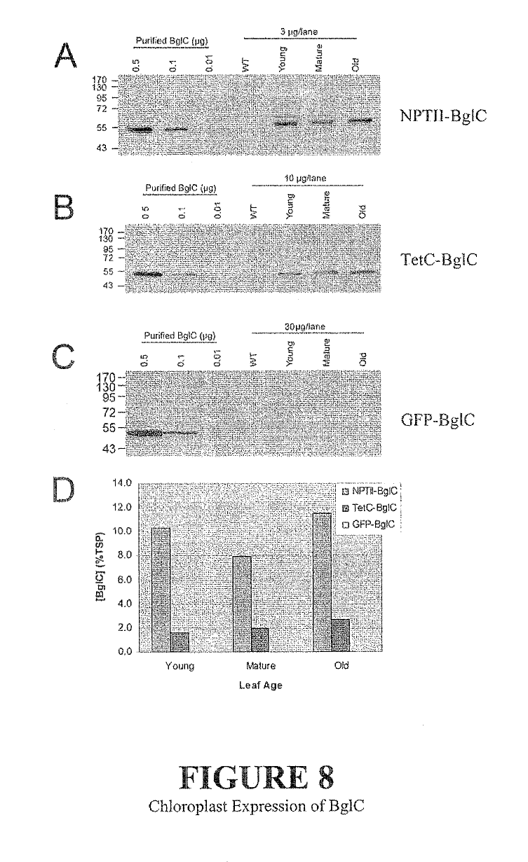

Chloroplast transformation vectors containing the bglC gene fused at its 5′ end to the TetC, NPTII, and GFP DB regions were constructed by PCR amplifying the bglC gene fused to these DB regions with the forward primers shown in Table 4 and a reverse primer BglC-rev also shown in Table 4. The nucleotides coding for the 5′ end of the bglC gene are underlined. Chloroplast transformation vectors were constructed and chloroplast-transformed tobacco generated using the methods described in Example 1. FIGS. 8A-8D shows that these experiments resulted in high-level NPTII-BglC production, with 8.0-11.6% of total soluble leaf protein present as NPTII-BglC

Beta-glucosidase (Bgl1 from Aspergillus niger) has been expressed previously in tobacco from the nuclear genome (Wei et al. (2004) Plant Biotechnol J 2: 341-350) and targeted to various subcellular locations, but the experiment described herein was the first report of β-glucosidase expression from the plastid genome. The best-expressing plant line produced by Wei et al. was only able to produce β-glucosidase at 2.3% TSP, while the best-expressing line of chloroplast-transformed tobacco expresses NPTII-BglC at 8.0-11.6% TSP, a 3.5- to 5-fold increase relative to the levels of β-glucosidase production reported by Wei et al. (2004).

### Example-3

This Example describes codon optimization of DB in fusion protein design.

The tobacco chloroplast genome and the E. coli (K12) genome both show a codon bias, with some codons used more often than others. In addition, some codons are preferentially used in the DB region of highly expressed native chloroplast genes and highly expressed native E. coli genes. Specifically, FIG. 9 and Table 1 show the codon usage frequencies of the 61 amino acid-encoding codons in the E. coli (K12) genome, with overall CUFs in black and DB CUFs (as calculated from codon usage in the DB regions of the nusA, arcB, rpoD, torS, ligA, topA, fryA, mutY, recQ, and rpoS genes) in gray. FIG. 10 and Table 2 show the codon usage frequency (CUF) of the 61 amino acid-encoding codons for all genes in the N. tabacum chloroplast genome (black bars) and for the DB regions of the genes encoding ten highly expressed proteins encoded in the N. tabacum chloroplast genome (i.e., the proteins encoded by the rbcL, psaA, psaB, psaC, psbA, psbB, psbC, psbD, psbE, and psbF genes; white bars).

Based on the codon usage frequency information, a GFP DB region was designed using the overall codon bias of E. coli (K12) (HF(EcTot)GFP) and using the DB region codon bias of E. coli (K12) (HF(EcDB)GFP), respectively. Table 5 shows the forward primers used to amplify GFP-Cel6A, HF(EcTot)GFP-Cel6A, and HF(EcDB)GFP-Cel6A. These genes, encoding the same amino acid sequence with changes only at the nucleotide level, were amplified by PCR using the primers shown in Table 5 in combination with primer Cel6A-TpsbA-rev (described in Example 1). In Table 5, the nucleotides encoding the 5′ end of the cel6A gene are underlined. The resulting PCR products were digested by NheI and NotI and inserted into pET expression vectors, then transformed into BL21(DE3) cells as described in Example 1. Protein expression is induced by IPTG addition as described in Example 1. FIGS. 11A-11B show data from E. coli expression of GFP-Cel6A, illustrating an improvement in GFP-Cel6A protein production due to codon optimization of the GFP DB region based on codon preferences in the downstream box regions of highly expressed native E. coli genes. Alteration of the GFP DB region to include codons preferentially used in the DB regions of highly expressed native E. coli genes allowed for an approximately 2-fold improvement in GFP-Cel6A expression from BL21(DE3) cells.

### Example-4

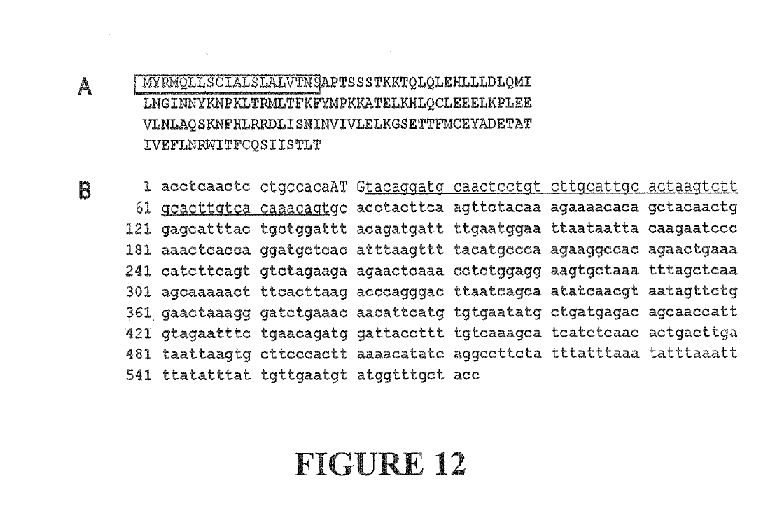

The IL-2 coding region was amplified from cDNA purchased from Invitrogen. The secretion signal peptide on the native gene (the first 20 amino acids) was deleted in order to accumulate the mature protein within chloroplast. Restriction sites Nde I and Pst I were introduced at the 5′ of IL-2 coding region, and a StuI site was introduced after the stop codon by PCR. For one transformation, the DNA codons for the first 13 amino acids (following the removal of the secretion signal peptide, FIG. 12) were modified (92.3%) to those used more frequently in DB of proteins in tobacco chloroplasts (Table 6).

PCR product of wild type and codon-modified IL-2 coding region were cloned into the PCR-cloning vector pGEM-Teasy (Promega) and sequenced, confirming that there were no mutations and the correct modifications were in place. Wild type and codon-modified IL-2 coding regions were digested by the restriction enzymes Nde I and Stu I and ligated into the tobacco chloroplast expression vector pBJF70. Recombinant clones were sequenced again to make sure the insertion sites were correct. New recombinant clones (pBJF70IL2 and pBJF70IL2M) were bombarded into tobacco chloroplast via the gene gun. The transformants were selected on RMOP plates containing 500 μg/ml spectinomycin as described in Gray et al. (Biotechnol Bioeng. (2009) 102 (4):1045-54). A total of 4 and 5 independent transformants were obtained for pBJF70IL2 and pBJF70IL2M respectively.

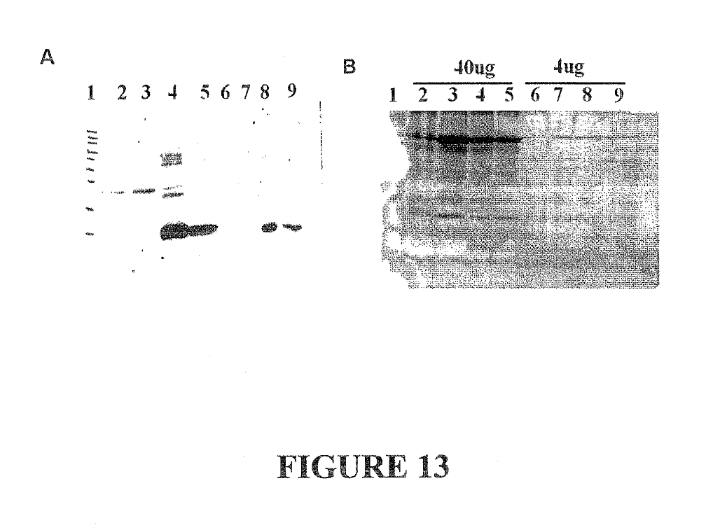

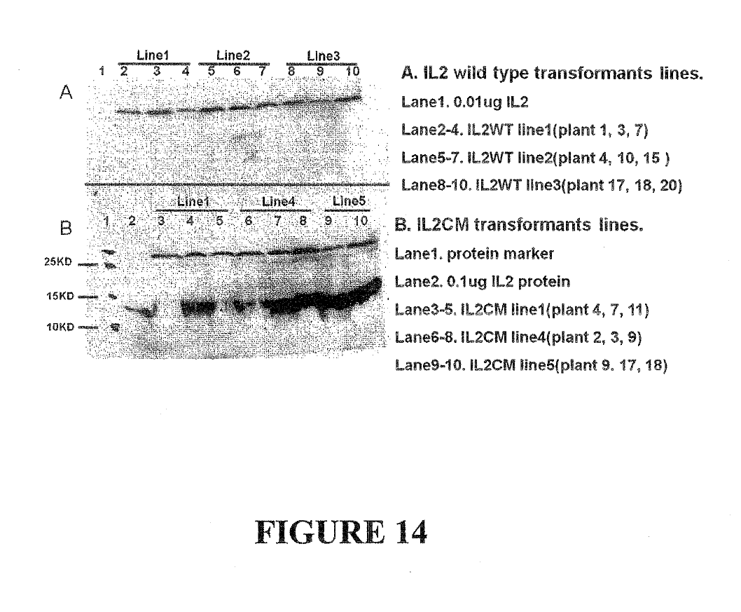

IL-2 protein accumulation was measured via an immunoblot analysis with commercially available monoclonal antibodies (FIGS. 13A-14) on several independent transformants.

TABLE 1
Codon usage frequency (CUF) table normalized
per 1000 codons for E. coli (Ec) arranged
by amino acid. Tot CUF is for all genes in
chloroplast and DB CUF is for downstream
box region of highly expressed genes.

| | Amino | | |
|---|---|---|---|
| Codon | Acid | EcTot CUF | EcDB CUF |
| GCG | Ala | 38.5 | 30.8 |
| GCC | Ala | 31.6 | 30.8 |
| GCT | Ala | 10.7 | 23.1 |
| GCA | Ala | 21.1 | 7.7 |
| CGA | Arg | 4.3 | 15.4 |
| AGA | Arg | 1.4 | 7.7 |
| CGT | Arg | 21.1 | 7.7 |
| AGG | Arg | 1.6 | 0 |
| CGC | Arg | 26 | 0 |
| CGG | Arg | 4.1 | 0 |
| AAT | Asn | 21.9 | 38.5 |
| AAC | Asn | 24.4 | 23.1 |
| GAT | Asp | 37.9 | 15.4 |
| GAC | Asp | 20.5 | 15.4 |
| TGT | Cys | 5.9 | 7.7 |
| TGC | Cys | 8 | 0 |
| CAA | Gln | 12.1 | 53.8 |
| CAG | Gln | 27.7 | 46.2 |
| GAA | Glu | 43.7 | 53.8 |
| GAG | Glu | 18.4 | 23.1 |
| GGT | Gly | 21.3 | 15.4 |
| GGC | Gly | 33.4 | 7.7 |
| GGA | Gly | 9.2 | 7.7 |
| GGG | Gly | 8.6 | 0 |
| CAT | His | 15.8 | 7.7 |
| CAC | His | 13.1 | 0 |
| ATT | Ile | 30.5 | 23.1 |
| ATC | Ile | 18.2 | 15.4 |
| ATA | Ile | 3.7 | 0 |
| CTG | Leu | 46.9 | 76.9 |
| TTA | Leu | 15.2 | 23.1 |
| CTT | Leu | 11.9 | 23.1 |
| TTG | Leu | 11.9 | 23.1 |
| CTC | Leu | 10.5 | 15.4 |
| CTA | Leu | 5.3 | 7.7 |
| AAA | Lys | 33.2 | 46.2 |
| AAG | Lys | 12.1 | 7.7 |
| ATG | Met | 24.8 | 7.7 |
| TTT | Phe | 19.7 | 23.1 |
| TTC | Phe | 15 | 0 |
| CCG | Pro | 26.7 | 15.4 |
| CCT | Pro | 8.4 | 15.4 |
| CCA | Pro | 6.6 | 0 |
| CCC | Pro | 6.4 | 0 |
| TCA | Ser | 7.8 | 23.1 |
| TCC | Ser | 5.5 | 23.1 |
| AGT | Ser | 7.2 | 7.7 |
| TCG | Ser | 8 | 7.7 |
| AGC | Ser | 16.6 | 0 |
| TCT | Ser | 5.7 | 0 |
| ACG | Thr | 11.5 | 30.8 |
| ACC | Thr | 22.8 | 23.1 |
| ACA | Thr | 6.4 | 7.7 |
| ACT | Thr | 8 | 0 |
| TGG | Trp | 10.7 | 15.4 |
| TAT | Tyr | 16.8 | 15.4 |
| TAC | Tyr | 14.6 | 7.7 |
| GTT | Val | 16.8 | 38.5 |
| GTC | Val | 11.7 | 15.4 |
| GTA | Val | 11.5 | 15.4 |
| GTG | Val | 26.4 | 7.7 |

TABLE 2
Codon usage frequency (CUF) table normalized
per 1000 codons for tobacco (Nt) arranged by
amino acid. Tot CUF is for all genes in
chloroplast and DB CUF is for downstream box
region of highly expressed genes.

| | Amino | | |
|---|---|---|---|
| Codon | Acid | NtTot CUF | NtDB CUF |
| GCT | Ala | 25.9 | 23.1 |
| GCA | Ala | 15.6 | 23.1 |
| GCC | Ala | 9.8 | 15.4 |
| GCG | Ala | 5.8 | 0 |
| CGT | Arg | 12.3 | 30.8 |
| CGA | Arg | 14.3 | 23.1 |
| AGA | Arg | 17.5 | 7.7 |
| AGG | Arg | 6.8 | 7.7 |
| CGC | Arg | 4 | 7.7 |
| CGG | Arg | 5 | 0 |
| AAT | Asn | 36.5 | 46.2 |
| AAC | Asn | 12.8 | 0 |
| GAT | Asp | 31.5 | 30.8 |
| GAC | Asp | 8.6 | 7.7 |
| TGT | Cys | 8 | 15.4 |
| TGC | Cys | 3 | 0 |
| CAA | Gln | 26 | 23.1 |
| CAG | Gln | 9 | 7.7 |
| GAA | Glu | 39.6 | 53.8 |
| GAG | Glu | 14.6 | 7.7 |
| GGA | Gly | 27.1 | 30.8 |
| GGT | Gly | 23.3 | 23.1 |
| GGC | Gly | 8 | 7.7 |
| GGG | Gly | 12.2 | 0 |
| CAT | His | 16.8 | 15.4 |
| CAC | His | 5.5 | 0 |
| ATT | Ile | 39.2 | 61.5 |
| ATA | Ile | 24.4 | 30.8 |
| ATC | Ile | 17.2 | 0 |
| TTA | Leu | 31 | 38.5 |
| TTG | Leu | 22.1 | 30.8 |
| CTT | Leu | 22.6 | 7.7 |
| CTA | Leu | 13.6 | 7.7 |
| CTC | Leu | 7.9 | 7.7 |
| CTG | Leu | 7.4 | 0 |
| AAA | Lys | 37.4 | 15.4 |
| AAG | Lys | 14.5 | 15.4 |
| ATG | Met | 24.5 | 0 |
| TTT | Phe | 34.2 | 46.2 |
| TTC | Phe | 20.6 | 0 |
| CCA | Pro | 12.1 | 30.8 |
| CCT | Pro | 17.1 | 7.7 |
| CCG | Pro | 5.6 | 7.7 |
| CCC | Pro | 7.3 | 0 |
| AGC | Ser | 5.4 | 30.8 |
| TCT | Ser | 22.1 | 15.4 |
| TCA | Ser | 15 | 15.4 |
| TCG | Ser | 8 | 15.4 |
| AGT | Ser | 14.9 | 0 |
| TCC | Ser | 12.8 | 0 |
| ACT | Thr | 20 | 30.8 |
| ACA | Thr | 15.1 | 23.1 |
| ACC | Thr | 10 | 23.1 |
| ACG | Thr | 5.4 | 15.4 |
| TGG | Trp | 17.2 | 30.8 |
| TAT | Tyr | 27.3 | 30.8 |
| TAC | Tyr | 7.7 | 0 |
| GTA | Val | 21.4 | 30.8 |
| GTT | Val | 20.1 | 23.1 |
| GTG | Val | 8.1 | 0 |
| GTC | Val | 7.2 | 0 |

TABLE 3
Primers and DBs Used in Example 1.

| SEQ | | |
|---|---|---|
| ID | Name | Sequence |
| 1 | ptDNA-fwd | ATCCCGGGGTTTCTCTCGCTTTTGG |
| 2 | ptDNA-rev | TAAAGCTTTGTATCGGCTA |
| 3 | lox-PpsbA-fwd | ATGCATCTGCAGATAACTTCGTATAATGTA |
| | | TGCTATACGAAGTTATCCCGGGCAACCCAC |
| | | TAGC |
| 4 | PpsbA-aadA-rev | AACCGCTTCACGAGCCATGGTAAAATCTTG |
| | | GTTTAT |
| 5 | PpsbA-aadA-fwd | ATAAACCAAGATTTTACCATGGCTCGTGAA |
| | | GCGGT |
| 6 | aadA-Trps16-rev | TAATTGAATTTCGGTTGATTATTTGCCAAC |
| | | TACCTT |
| 7 | aadA-Trps16-fwd | AAGGTAGTTGGCAAATAATCAACCGAAATT |
| | | CAATTA |
| 8 | Trps16-lox-rev | ATGCATAACTTCGTATAGCATACATTATAC |
| | | GAAGTTATACGGAATTCAATGGAAGC |
| 9 | trnIint-fwd | CTGGGGTGACGGAGGGAT |
| 10 | rmvNdeI1 | CTGACGTCTAAGAAACCA |
| 11 | rmvNdeI2 | TACTGAGAGTGCACCAAATGCGGTGTGAAA |
| 12 | rmvNdeI3 | TTTCACACCGCATTTGGTGCACTCTCAGTA |
| 13 | rmvNdeI4 | ATGGGCCCGCTATGCCAAAAGC |
| 14 | T7-fwd | CTGCAGGCGCGCCGGGAGACCACAACGGTT |
| | | TCCCACTAGAAATAA |
| 15 | T7-rev | GCTAGCCATATGTATATC |
| 16 | GFPCe16A-fwd | ATGCTAGCGGCAAGGGCGAGGAACTGTTCA |
| | | CTGGCGTGGTCCCAATCAATGATTCTCCGT |
| | | TCTAC |
| 17 | Ce16A-TpsbA-rev | ATAGACTAGGCCAGGATCGCGGCCGCTCAG |
| | | CTGGCGGCGCAGGT |
| 18 | Ce16A-TpsbA-fwd | ACCTGCGCCGCCAGCTGAGCGGCCGCGATC |
| | | CTGGCCTAGTCTAT |
| 19 | TpsbA-rev | ATGCTAGCTGCAGAAAAAGAAAGGAGCAAT |
| 20 | C6probe-fwd | AGTAACGAGTGGTGCGACC |
| 21 | TetCCe16A-fwd | ATGCTAGCAAAAATCTGGATTGTTGGGTCG |
| | | ACAATGAAGAAGATATAAATGATTCTCCGT |
| | | TCTAC |
| 22 | NPTIICe16A-fwd | ATGGCTAGCATTGAACAAGATGGATTGCAC |
| | | GCAGGTTCTCCGGCCGCTAATGATTCTCCG |
| | | TTCTAC |
| 23 | probe-fwd | ATAGTATCTTGTACCTGA |
| 24 | Cel6Aint-rev | TGCTGTGGTTGCCGCAGT |
| 25 | Iprobe-fwd | CACAGGTTTAGCAATGGG |
| 26 | Iprobe-rev | GAAGTAGTCAGATGCTTC |

TABLE 4
Primers for Amplification of DB-BglC
Fusions for Chloroplast Expression

| D13 Region | |
|---|---|
| fused to BglC | Primer Sequence |
| TetC | CATATGGCTAGCAAAAATCTGGATTGTTGGGTCGAC |
| | AATGAAGAAGATATAACCTCGCAATCGACGACT |
| | (SEQ ID NO: 27) |
| NPTII | CATATGGCTAGCATTGAACAAGATGGATTGCACGCA |
| | GGTTCTCCGGCCGCTACCTCGCAATCGACGACT |
| | (SEQ ID NO: 28) |
| GFP | CATATGGCTAGCGGCAAGGGCGAGGAACTGTTCACT |
| | GGCGTGGTCCCAATCACCTCGCAATCGACGACT |
| | (SEQ ID NO: 29) |
| BglC-rev | ATGCGGCCGCTATTCCTGTCCGAAGAT |
| | (SEQ ID NO: 30) |

TABLE 5
Forward Primers for Amplification
of Codon-Optimized GFP-Cel6A

| DB Region | |
|---|---|
| fused to Cel6A | Primer Sequence |
| GFP | ATCATATGGCTAGCGGCAAGGGCGAGGAACTGTTC |
| | ACTGGCGTGGTCCCAATCAATGATTCTCCGTTCTA |
| | C |
| | (SEQ ID NO: 31) |
| HF(EcTot)GFP | ATCATATGGCTAGCGGCAAAGGCGAAGAACTGTTT |
| | ACCGGCGTGGTGCCGATTAATGATTCTCCGTTCTA |
| | C |
| | (SEQ ID NO: 32) |
| HF(EcDB)GFP | ATCATATGGCTAGCGGTAAAGGTGAAGAACTGTTT |
| | ACGGGTGTTGTTCCGATTAATGATTCTCCGTTCTA |
| | C |
| | (SEQ ID NO: 33) |
| | KNLDCWVDNEEDI |
| | SEQ ID NO: 34 |
| | IEQDGLHAGSPAA |
| | SEQ ID NO: 35 |
| | AS KNLDCWVDNEEDI |
| | SEQ ID NO: 36 |
| | AS IEQDGLHAGSPAA |
| | SEQ ID NO: 37 |
| | ATG AAA AAT CTG GAT TGT TGG GTC GAC |
| | AAT GAA GAA GAT ATA GAT GTT ATA TTA |
| | AAA AAG AGT ACA ATT TTA AAT TTA GAT |
| | ATT AAT AAT |
| | SEQ ID NO: 38 |
| | MKNLDCWVDNEEDIDVILKKSTILNLDINN |
| | SEQ ID NO: 39 |
| | ATG GCT AGC ATT GAA CAA GAT GGA TTG |
| | CAC GCA GGT TCT CCG GCC GCT TGG GTG |
| | GAG AGG CTA TTC GGC TAT GAC TGG GCA |
| | CAA CAG ACA |
| | SEQ ID NO: 40 |
| | MASIEQDGLHAGSPAAWVERLFGYDWAQQT |
| | SEQ ID NO: 41 |

TABLE 6
Codon modifications to DB of mature IL-2.

| 13 first | original | | modified | |
|---|---|---|---|---|
| aa of IL-2 | codon | DB CUF | codon | DB CUF |
| Ala | gca | 23.1 | gca | 23.1 |
| Pro | cct | 7.7 | cca | 30.8 |
| Thr | act | 30.8 | act | 30.8 |
| Ser | tca | 15.4 | agc | 30.8 |
| Ser | agt | 0 | agc | 30.8 |
| Ser | tct | 15.4 | agc | 30.8 |
| Thr | aca | 23.1 | act | 30.8 |
| Lys | aag | 15.4 | aag | 15.4 |
| Lys | aaa | 15.4 | aaa | 15.4 |
| Thr | aca | 23.1 | act | 30.8 |
| Gln | cag | 7.1 | caa | 23.1 |
| Leu | cta | 7.7 | cta | 7.7 |
| Gln | caa | 23.1 | caa | 23.1 |

1. An isolated nucleic acid molecule, comprising at least 10 contiguous codons from the downstream box region of the tetC gene as set forth in SEQ ID NO: 38.

2. The isolated nucleic acid molecule of claim 1, wherein said downstream box region of the tetC gene encodes the peptide sequence of KNLDCWVDNEEDI (SEQ ID NO: 34).

3. The isolated nucleic acid molecule of claim 1, encoding the peptide sequence of ASKNLDCWVDNEEDI (SEQ ID NO: 36).

4. An isolated nucleic acid molecule, comprising at least 10 contiguous codons from the downstream box region of the neo gene as set forth in SEQ ID NO: 40.

5. The isolated nucleic acid molecule of claim 1, wherein said downstream box region of the neo gene encodes the peptide sequence of IEQDGLHAGSPAA (SEQ ID NO: 35).

6. The isolated nucleic acid molecule of claim 1, encoding the peptide sequence of ASIEQDGLHAGSPAA (SEQ ID NO: 37).

7. A nucleic acid construct for expression of a protein in plastids, comprising an isolated nucleic acid molecule according to any one of claims 1-2 or 4-5, inserted in-frame and immediately downstream of the start codon of the coding sequence for said protein, and wherein said protein is not TetC or NPTII.

8-9. (canceled)

10. An expression vector, comprising the nucleic acid construct of claim 7.

11. An isolated nucleic acid molecule, comprising at least 10 contiguous codons from the downstream box region of a gene of interest, wherein said at least 10 contiguous codons have been codon-optimized for protein expression in an organism or organelle based on the codon usage frequencies of the DB regions of highly expressed native genes in said organism or organelle.

12. An isolated nucleic acid construct for expression of a protein in an organism or organelle, comprising the nucleic acid molecule of claim 11, placed in-frame and immediately downstream of the start codon of the coding sequence encoding said protein.

13. The nucleic acid construct of claim 12, wherein said downstream box region is native to said coding sequence.

14. The nucleic acid construct of claim 12, wherein said downstream box region is derived from a heterologous gene.

15. The nucleic acid construct of claim 12, wherein said organism is selected from plant, bacteria or algae.

16. The nucleic acid construct of claim 12, wherein said organelle is plastid.

17. An expression vector, comprising the nucleic acid construct of claim 12.

18. A method for expression of a protein in plastids, comprising generating a nucleic acid construct by inserting in-frame and immediately downstream of the start codon of the coding sequence for said protein, a nucleic acid molecule according to any one of claims 1-2 or 4-5, transforming said nucleic acid construct into plastids, and expressing said protein in said plastids.

19. The method of claim 18, wherein said plastids are plastids of tobacco.

20. A method for expression of a protein in an organism or organelle, comprising generating a nucleic acid construct by inserting in-frame and immediately downstream of the start codon of the coding sequence for said protein, a downstream box sequence that has been codon-optimized for expression in said organism or organelle, transforming said nucleic acid construct into said organism or organelle, and expressing said protein in said organism or organelle.

21. A method for improving expression of a protein in an organism or organelle, comprising optimizing the downstream box region of the coding sequence for said protein for expression in said organism or organelle, transforming the codon-optimized coding sequence into said organism or organelle, and expressing said protein in said organism or organelle.

22. The method of claim 20 or 21, wherein said organism is plant, bacteria or algae.

23. The method of claim 20 or 21, said organelle is plastid.

24. A transgenic plant, comprising the nucleic acid construct of claim 7 integrated in the plastid genome.

25-28. (canceled)

29. A transgenic plant, comprising a nucleic acid construct according to claim 12 or 16.

30-33. (canceled)


SEQUENCE LISTING (43 sequences)

SEQ ID NO: 1 - DNA, 25 bp, Artificial Sequence
  Synthetic oligonucleotide primer
atcccggggtttctctcgcttttgg

SEQ ID NO: 2 - DNA, 19 bp, Artificial Sequence
  Synthetic oligonucleotide primer
taaagctttgtatcggcta

SEQ ID NO: 3 - DNA, 64 bp, Artificial Sequence
  Synthetic oligonucleotide primer
atgcatctgcagataacttcgtataatgtatgctatacgaagttatcccgggcaacccactagc

SEQ ID NO: 4 - DNA, 36 bp, Artificial Sequence
  Synthetic oligonucleotide primer
aaccgcttcacgagccatggtaaaatcttggtttat

SEQ ID NO: 5 - DNA, 35 bp, Artificial Sequence
  Synthetic oligonucleotide primer
ataaaccaagattttaccatggctcgtgaagcggt

SEQ ID NO: 6 - DNA, 36 bp, Artificial Sequence
  Synthetic oligonucleotide primer
taattgaatttcggttgattatttgccaactacctt

SEQ ID NO: 7 - DNA, 36 bp, Artificial Sequence
  Synthetic oligonucleotide primer
aaggtagttggcaaataatcaaccgaaattcaatta

SEQ ID NO: 8 - DNA, 56 bp, Artificial Sequence
  Synthetic oligonucleotide primer
atgcataacttcgtatagcatacattatacgaagttatacggaattcaatggaagc

SEQ ID NO: 9 - DNA, 18 bp, Artificial Sequence
  Synthetic oligonucleotide primer
ctggggtgacggagggat

SEQ ID NO: 10 - DNA, 18 bp, Artificial Sequence
  Synthetic oligonucleotide primer
ctgacgtctaagaaacca

SEQ ID NO: 11 - DNA, 30 bp, Artificial Sequence
  Synthetic oligonucleotide primer
tactgagagtgcaccaaatgcggtgtgaaa

SEQ ID NO: 12 - DNA, 30 bp, Artificial Sequence
  Synthetic oligonucleotide primer
tttcacaccgcatttggtgcactctcagta

SEQ ID NO: 13 - DNA, 22 bp, Artificial Sequence
  Synthetic oligonucleotide primer
atgggcccgctatgccaaaagc

SEQ ID NO: 14 - DNA, 45 bp, Artificial Sequence
  Synthetic oligonucleotide primer
ctgcaggcgcgccgggagaccacaacggtttcccactagaaataa

SEQ ID NO: 15 - DNA, 18 bp, Artificial Sequence
  Synthetic oligonucleotide primer
gctagccatatgtatatc

SEQ ID NO: 16 - DNA, 65 bp, Artificial Sequence
  Synthetic oligonucleotide primer
atgctagcggcaagggcgaggaactgttcactggcgtggtcccaatcaatgattctccgttctac

SEQ ID NO: 17 - DNA, 44 bp, Artificial Sequence
  Synthetic oligonucleotide primer
atagactaggccaggatcgcggccgctcagctggcggcgcaggt

SEQ ID NO: 18 - DNA, 44 bp, Artificial Sequence
  Synthetic oligonucleotide primer
acctgcgccgccagctgagcggccgcgatcctggcctagtctat

SEQ ID NO: 19 - DNA, 31 bp, Artificial Sequence
  Synthetic oligonucleotide primer
atgctagctgcagaaaaagaaaggagcaata

SEQ ID NO: 20 - DNA, 18 bp, Artificial Sequence
  Synthetic oligonucleotide primer
gtaacgagtggtgcgacc

SEQ ID NO: 21 - DNA, 65 bp, Artificial Sequence
  Synthetic oligonucleotide primer
atgctagcaaaaatctggattgttgggtcgacaatgaagaagatataaatgattctccgttctac

SEQ ID NO: 22 - DNA, 66 bp, Artificial Sequence
  Synthetic oligonucleotide primer
atggctagcattgaacaagatggattgcacgcaggttctccggccgctaatgattctccgttctac

SEQ ID NO: 23 - DNA, 18 bp, Artificial Sequence
  Synthetic oligonucleotide primer
atagtatcttgtacctga

SEQ ID NO: 24 - DNA, 18 bp, Artificial Sequence
  Synthetic oligonucleotide primer
tgctgtggttgccgcagt

SEQ ID NO: 25 - DNA, 18 bp, Artificial Sequence
  Synthetic oligonucleotide primer
cacaggtttagcaatggg

SEQ ID NO: 26 - DNA, 18 bp, Artificial Sequence
  Synthetic oligonucleotide primer
gaagtagtcagatgcttc

SEQ ID NO: 27 - DNA, 69 bp, Artificial Sequence
  Synthetic oligonucleotide primer
catatggctagcaaaaatctggattgttgggtcgacaatgaagaagatataacctcgcaatcgacgact

SEQ ID NO: 28 - DNA, 69 bp, Artificial Sequence
  Synthetic oligonucleotide primer
catatggctagcattgaacaagatggattgcacgcaggttctccggccgctacctcgcaatcgacgact

SEQ ID NO: 29 - DNA, 69 bp, Artificial Sequence
  Synthetic oligonucleotide primer
catatggctagcggcaagggcgaggaactgttcactggcgtggtcccaatcacctcgcaatcgacgact

SEQ ID NO: 30 - DNA, 27 bp, Artificial Sequence
  Synthetic oligonucleotide primer
atgcggccgctattcctgtccgaagat

SEQ ID NO: 31 - DNA, 71 bp, Artificial Sequence
  Synthetic oligonucleotide primer
atcatatggctagcggcaagggcgaggaactgttcactggcgtggtcccaatcaatgattctccgttctac

SEQ ID NO: 32 - DNA, 71 bp, Artificial Sequence
  Synthetic oligonucleotide primer
atcatatggctagcggcaaaggcgaagaactgtttaccggcgtggtgccgattaatgattctccgttctac

SEQ ID NO: 33 - DNA, 71 bp, Artificial Sequence
  Synthetic oligonucleotide primer
atcatatggctagcggtaaaggtgaagaactgtttacgggtgttgttccgattaatgattctccgttctac

SEQ ID NO: 34 - protein, 13 aa, Artificial Sequence
  Synthetic peptide
KNLDCWVDNEEDI

SEQ ID NO: 35 - protein, 13 aa, Artificial Sequence
  Synthetic peptide
IEQDGLHAGSPAA

SEQ ID NO: 36 - protein, 15 aa, Artificial Sequence
  Synthetic peptide
ASKNLDCWVDNEEDI

SEQ ID NO: 37 - protein, 15 aa, Artificial Sequence
  Synthetic peptide
ASIEQDGLHAGSPAA

SEQ ID NO: 38 - DNA, 90 bp, Artificial Sequence
  Nucleotide sequence representing the first 30 codons of the TetC gene
atgaaaaatctggattgttgggtcgacaatgaagaagatatagatgttatattaaaaaagagtacaattttaaatttagatattaataat

SEQ ID NO: 39 - protein, 30 aa, Artificial Sequence
  Synthetic peptide
MKNLDCWVDNEEDIDVILKKSTILNLDINN

SEQ ID NO: 40 - DNA, 90 bp, Artificial Sequence
  Nucleotide sequence representing the first 30 codons of the neo gene
atggctagcattgaacaagatggattgcacgcaggttctccggccgcttgggtggagaggctattcggctatgactgggcacaacagaca

SEQ ID NO: 41 - protein, 30 aa, Artificial Sequence
  Synthetic peptide
MASIEQDGLHAGSPAAWVERLFGYDWAQQT

SEQ ID NO: 42 - protein, 152 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRRDLISNINVIVLELKGSETTFMCEYADETATIVEFLNRWITFCQSIISTLT

SEQ ID NO: 43 - DNA, 573 bp, Homo sapiens
acctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacc